(19) United States
(12) Patent Application Publication
(10) Pub. No.: US 2014/0349931 A1
(43) Pub. Date: Nov. 27, 2014

(54) DETECTION AND TREATMENT OF BREAST CANCER

(71) Applicants: The European Molecular Biology Laboratory, Heidelberg (DE); UCL Business PLC, London (GB); Fahy Gurteen Laboratories Ltd., Dublin (IE)

(72) Inventors: Jan Ellenberg, Heidelberg (DE); Beate Neumann, Heidelberg (DE); Marco Loddo, London (GB); Gareth Williams, London (GB); Kai Stoeber, London (GB)

(73) Assignees: THE EUROPEAN MOLECULAR BIOLOGY LABORATORY, Heidelberg (DE); FAHY GURTEEN LABORATORIES LTD., Dublin (IE); UCL BUSINESS PLC, London (GB)

(21) Appl. No.: 14/366,878

(22) PCT Filed: Dec. 20, 2012
(86) PCT No.: PCT/GB2012/053223

§ 371 Date: Jun. 19, 2014

(30) Foreign Application Priority Data
Dec. 20, 2011 (GB) 1121924.3

(51) Int. Cl. C12Q 1/68; A61K 38/30; A61K 45/06; G01N 33/574

(52) U.S. Cl. CPC C12Q 1/6886; G01N 33/57415; A61K 38/30; A61K 45/06; C12Q 2600/118; C12Q 2600/154
USPC 514/8.6; 435/6.11; 436/501; 506/2; 506/9; 435/7.94; 435/7.1; 530/399

(57) ABSTRACT

The present invention describes methods for determining the risk that a breast precursor lesion will progress to invasive breast cancer and/or the risk of recurrent non-invasive disease in a patient, comprising detecting the presence and/or level of PAPPA and/or PAPPA functional activity in a breast tissue sample obtained from the patient, wherein if PAPPA is not present, or is present at a reduced amount compared to a control, there is the risk of progression to invasive cancer and/or the risk or recurrent disease.

The present invention also enables the chemosensitisation of mitotically delayed breast cancer cells to anti-proliferative agents, preferably anti-mitotic agents, by restoring normal progression through mitosis. In this embodiment a first drug is applied to release breast cancer cells from the mitotic block and, sequentially, a second drug affecting proliferating cells is administered for cancer cell killing.

### FIELD OF THE INVENTION

This invention relates to the use of specific biological markers for the prognostic assessment of proliferative lesions in breast tissue, and for identifying the risk of proliferative lesions progressing to invasive breast cancer and/or the risk of developing recurrent disease, and subsequent treatment.

### BACKGROUND OF THE INVENTION

Neoplasms and cancer are abnormal growths of cells. Cancer cells rapidly reproduce despite restriction of space, nutrients shared by other cells, or signals sent from the body to stop reproduction. Cancer cells are often shaped differently from healthy cells, do not function properly, and can spread into many areas of the body. Abnormal growths of tissue, called tumours, are clusters of cells that are capable of growing and dividing uncontrollably. Tumours can be benign (noncancerous) or malignant (cancerous). Benign tumours tend to grow slowly and do not spread. Malignant tumours can grow rapidly, invade and destroy nearby normal tissues, and spread throughout the body. Precursor lesions such as pre-invasive lesions or proliferative lesions with uncertain malignant potential represent abnormal growths of tissue which can behave in a benign fashion or alternatively progress to an invasive malignant cancer. Malignant cancers can be both locally invasive and metastatic.

Breast cancer is an example of a common cancer and is a complex disease due to its morphological and biological heterogeneity, its tendency to acquire chemo-resistance and the existence of several molecular mechanisms underline its pathogenesis. Half of women who receive loco-regional treatment for breast cancer will never relapse, whereas the other half will eventually die from metastatic disease. It is therefore imperative to distinguish clearly between those two groups of patients for optimal clinical management. There is also an urgent need to identify prognostic markers for the identification of patients with precursor lesions who are at high risk of progression to invasive breast cancer. Unfortunately however, prognostic markers for breast cancer are limited.

Treatment for breast cancer can vary depending on the stage of progression of the cancer. Breast cancer is often detected at an early stage, when the cancer is said to be pre-invasive, for example in ductal carcinoma in situ (DCIS), where cancer cells or non-cancerous abnormal cells have not invaded neighbouring normal tissue. A complexity for treatment is that not all pre-invasive cancers will progress to become invasive (or metastatic), for example as in DCIS, and so not all patients need to be treated in the same way. A difficulty is that at present it is difficult to differentiate between patients who have a cancer (or abnormal cells) that will remain pre-invasive, and those who will progress to invasive cancer.

### SUMMARY OF THE INVENTION

The present invention is based on the finding that Pregnancy-Associated Plasma Protein A (PAPPA) is required for normal progression through mitosis, and that PAPPA silencing is highly prevalent in invasive breast cancer and pre-invasive lesions predisposed to becoming invasive. Therefore, the present invention provides a very important understanding to the biological causes of breast cancer, and allows consequent detection and treatment of breast cancer to be made in a more focussed and effective way. The understanding that PAPPA is required for normal progression through mitosis, and that the loss of its expression or impaired functioning contributes significantly to the cancerous state, and in particular progression from pre-invasive to invasive cancer, allows detection of breast cancer to be made by monitoring PAPPA levels, and treatment to be given by targeting therapies for increasing endogenous PAPPA levels.

In proliferative lesions within breast tissue, cells that have no or reduced PAPPA levels or activity and are stalled in mitosis are pre-disposed to developing an invasive character. Accordingly, the present invention allows patients who are pre-disposed to developing invasive breast cancer to be identified, either on the basis of PAPPA levels/functional activity, or by identifying cells with the delayed mitotic phenotype. Proliferative lesions within breast tissue samples can therefore be classified as either likely to remain non-invasive, or pre-disposed to becoming invasive.

According to a first aspect of the invention, there is a method for determining the risk of progression of a proliferative lesion to invasive breast cancer and/or the risk of recurrent non-invasive disease in a patient, comprising detecting the presence and/or level of PAPPA in a breast tissue sample obtained from the patient, wherein if PAPPA is not present, or is present at a reduced level compared to a control, there is the risk of progression to invasive cancer and/or risk of recurrent disease.

According to a second aspect of the invention, there is a method for determining the risk of progression of a proliferative lesion to invasive breast cancer and/or the risk of recurrent non-invasive disease in a patient, comprising detecting the presence of loss of function-related genetic alterations in the PAPPA gene or its regulatory or promoter sequences in a sample obtained from the patient, wherein if genetic alterations are present, there is the risk of progression to invasive cancer and/or risk of recurrent disease.

According to a third aspect of the invention, there is a method of determining the risk of progression of a proliferative lesion to invasive breast cancer and/or the risk of recurrent non-invasive disease in a patient, comprising identifying the proportion of mitotic cells in a breast tissue sample obtained from the patient that are in prophase or pro-metaphase, wherein if the proportion of cells in prophase or pro-metaphase is 30% or more, this indicates a risk of progression to invasive breast cancer and/or the risk of recurrent disease.

According to a fourth aspect of the invention, there is the use of H3S10ph immuno-detection to determine the risk of progression of a proliferative lesion to invasive breast cancer in a patient and/or risk of recurrent disease.

According to a fifth aspect of the invention, a therapeutic regimen for treating or preventing breast cancer in a patient comprises: (i) administering a first drug which releases mitotically delayed cells; and (ii) sequentially administering a second drug which is a chemotherapeutic agent.

According to a sixth aspect, the invention provides a chemotherapeutic agent that targets molecular events during cell division, for use in the treatment or prevention of breast cancer, wherein the chemotherapeutic is to be administered to a patient who has been prior treated with a therapeutic agent that releases a cell from mitotic delay.

### DESCRIPTION OF THE DRAWINGS

The invention is described with reference to the accompanying figures, wherein:

FIG. 1 shows mitotic phase distribution in human cancers. 1a shows representative images identifying distinct mitotic phases by H3S10ph immunolabelling in tissue sections of surgical biopsy specimens (1000× magnification). 1b shows pie charts showing the percentage of mitotic cells assigned to each mitotic phase in normal breast (n=5 patients), lymphoma (n=29 patients) and in breast cancer (n=156 patients), lung cancer (n=30 patients), bladder cancer (n=27 patients) and colon cancer (n=41 patients). 1c shows representative cases of breast cancer (400× magnification; scale bar 50 μm) and non-invasive ductal carcinoma in situ (DCIS; 400× magnification; scale bar 50 μm) which show a high frequency of early mitotic figures compared to bladder cancer (1000× magnification; scale bar 20 μm) and other cancer types (not shown);

FIG. 2 shows specificity of phosphohistone H3 (H3S10ph) as a mitotic marker. HeLa Kyoto cells were synchronised at the G1/S transition by double-thymidine block and in prometaphase by treatment with the Plk-1 inhibitor BI2536 (SelleckChem) at 5 μM. Asynchronously proliferating (UT), thymidine-arrested, and BI2536-treated cells were immuno-labelled for phosphohistone H3 (H3S10ph). Phosphohistone H3 was not detected in thymidine-arrested cells by immunofluorescence or chromogenic staining, whereas BI2536-treated showed an enrichment of prometaphase cells (arrows) positive for H3S10ph. Panels on the right show flow cytometric analysis of DNA content;

FIG. 3 is a Receiver Operating Characteristic curve for prophase/prometaphase fraction applying a minimum mitotic cell count of n=5;

FIG. 4 shows the distribution of prophase/prometaphase fraction in breast cancer, DCIS and other cancers (pooled);

FIG. 5 shows enrichment of early mitotic figures in breast cancer. 5a is a box plot showing the percentage of mitotic cells in prophase/prometaphase in a range of human cancers. Breast cancer is characterised by a higher proportion of mitotic cells in prophase/prometaphase compared to other tumour types (P<0.0001). The median (solid black line), interquartile range (boxed) and range (enclosed by lines) are shown. Outlying cases are depicted as isolated points. 5b shows photomicrographs of representative cases of normal breast, breast cancer and other types of cancer immuno-labelled for phosphohistone H3 (H3S10ph) and assigned to distinct mitotic phases (see key below; 1000× magnification, scale bar 20 μm),

FIG. 6 shows that acquisition of the mitotic delay phenotype occurs early in multi-step mammary tumour progression. 6a shows photomicrographs of representative cases of non-invasive ductal carcinoma in situ (DCIS) immuno-labelled for phosphohistone H3 (H3S10ph) showing normal mitotic phase distribution (left panel) and a high proportion of mitotic cells in prophase/prometaphase (right panel) (1000× magnification; scale bar 20 μm). 6b is a bar chart showing the percentage of cases of normal breast, DCIS and breast cancer exhibiting prophase/prometaphase delay. Cases are defined as delayed if the proportion of mitotic cells in prophase/prometaphase is at least one third. This cut-point was chosen to allow the proportion of specimens in the combined group of other malignancies properly classified as non-delayed (94.1%) to be approximately equal to the proportion of breast cancer specimens properly classified as delayed (94.9%). 6c is a box plot showing the percentage of mitotic cells in prophase/prometaphase in normal breast, DCIS and breast cancer. There is a trend for increasing early mitotic delay during transition from normal breast to invasive breast cancer (P<0.001),

FIG. 7 shows selection of MitoCheck prophase/prometaphase class genes for further study. The genome-wide MitoCheck RNAi screen was performed by time-lapse fluorescence microscopy of live HeLa Kyoto cells stably expressing a fluorescent chromosome marker (Histone 2B-GFP) (1). Automated fluorescence imaging of siRNA transfected cells was followed by computational phenotyping of mitotic stages and mitotic defects from digital images (2). The time-resolved phenoprints were analysed to cluster candidate genes by phenotype (3). Data mining of the MitoCheck prophase/prometaphase class genes revealed 41 genes linked to early mitotic phase progression (4, 5). Several exclusion criteria (for example, no massive cell death as a secondary phenotype) were used to narrow the list of candidates to seven genes whose knock down caused a sharp increase in the percentage of mitotic cells in prophase/prometaphase (6, 7);

FIG. 8a shows time-resolved heat maps for seven candidate genes identified in the genome-wide MitoCheck screen in HeLa cells, which show as a primary phenotype prometaphase arrest/delay followed by secondary phenotypes (binuclear, polylobed, grape-shaped and cell death). FIG. 8b shows that knock down of all seven candidate genes caused a significant increase in the percentage of mitotic cells in prophase/prometaphase;

FIG. 9 shows PAPPA silencing through promoter methylation is linked to mitotic delay in breast cancer. 9a is a heat map showing the promoter methylation status (determined by MethyLight assay as percentage methylated reference gene [PMR]) of the seven MitoCheck candidate genes in normal breast (n=30 patients), low-grade (n=39 patients) and high-grade (n=36 patients) non-invasive DCIS breast lesions, and in invasive breast cancer (n=173 patients). PMR values are presented by coloured bars as shown in the key below the panel. 9b is a stacked bar chart showing normal breast (n=30 patients), DCIS (n=75 patients) and breast cancer (n=173 patients) cases ranked by PAPPA promoter methylation level. 9c shows PAPP-A protein expression in normal proliferating (pregnant) breast, DCIS and breast cancer cases in relation to mitotic delay phenotype and PAPPA promoter methylation status (1000× magnification; scale bar 10 μm). PAPPA antibody specificity was confirmed by peptide blocking (lower right panel). 9d is a heat map showing the promoter methylation status of the seven MitoCheck candidate genes in cultured primary, immortalised, and transformed breast cells. 9e shows detection of PAPPA protein by western blot in the cultured breast cells described in panel (d). 9f is a stacked bar chart showing the percentage of mitotic cells in the cultured breast cells described in panel (d) assigned to distinct mitotic phases;

FIG. 10 shows characterisation of rabbit polyclonal antibody raised against PAPPA. 10a is a Western blot detection of endogenous PAPPA in whole cell extracts prepared from MCF10A and BT549 cells. Pre-incubation with a blocking peptide confirmed the specificity of the PAPPA antibody, while the pre-immune serum did not detect endogenous PAPPA. 10b is a Western blot analysis of whole cell extracts prepared from untreated (UT), ZMPSTE24 overexpressing (CO) and PAPPA overexpressing (PAPPA+) T47D cells with PAPPA antibody. Notably T47D cells show PAPPA promoter hypermethylation and do not express the endogenous protein. 10c shows immunoprecipitation of PAPP-A protein from cell culture medium obtained from PAPPA overexpressing T47D cell populations (PAPPA+) 72 hour post-transfection. PAPPA protein was immuno-precipitated with a commercially supplied PAPPA rabbit polyclonal antibody (DAKO) and detected by Western blot using the in-house raised PAPPA antibody. FT: flow-through following overnight incubation with antibody coated beads; Elution: bound proteins eluted from beads with loading buffer;

FIG. 11 shows experimental manipulation of PAPPA expression controls transit through early mitosis. 11a shows PAPPA transcript levels were knocked down (KD) by RNAi in BT549 cells, resulting in PAPPA protein depletion compared to untreated (UT) and control-transfected (CO) cells. 11b shows PAPP-A protein levels were restored in T47D cells (PAPPA gene epigenetically silenced by promoter methylation) after transfection with a PAPPA expression construct (PAPPA+). T47D cells were control-transfected (CO) with an expression construct for an unrelated metalloproteinase (ZMPSTE24), which—like PAPPA—is a member of the metzincin family. 11c shows RNAi against PAPPA in BT549 cells was associated with a marked increase in early mitotic figures as determined by phosphohistone H3 (H3S10ph) immuno-labelling of cytospin preparations, while exogenously expressed PAPPA restored normal mitotic phase distribution in T47D cells. 11d shows the indicated time points cell number was measured in UT, CO and KD BT549 cells. 11e shows the DNA content of UT, CO and KD BT549 cells 48 and 72 hours post-transfection.

FIG. 12 shows RNAi specificity control and rescue experiments in BT549 breast cancer cells. 12a shows PAPPA transcript levels in untreated (UT) cells, PAPPA-siRNA 104028 (KD28) or PAPPA-siRNA 10042 (KD42) transfected cells, and in cells transfected with PAPPA RNAi rescue construct in the presence of PAPPA siRNA (PAPPA+^{mut}/KD28 or PAPPA+^{mut}/KD42) relative to cells transfected with a ZMPSTE24 expression construct (CO). 12b is a Western blot analysis of PAPP-A protein in whole cell extracts prepared from UT, CO, KD28, KD42, PAPPA+^{mut}/KD28 and PAPPA+^{mut}/KD42 cells 72 hours post-transfection. 12c shows UT, CO, KD28, KD42, PAPPA+^{mut}/KD28 and PAPPA+^{mut}/KD42 cells were cytospun onto glass slides and mitotic cells were detected by phosphohistone H3 (H3S10ph) immuno-labelling. 12d is a stacked bar chart showing the percentage of UT, CO, KD28, KD42, PAPPA+^{mut}/KD28 and PAPPA+^{mut}/KD42 cells in distinct mitotic phases;

FIG. 13 shows PAPPA expression levels affect the invasive capacity of breast cancer cell lines. 13a shows that PAPPA expression in BT549 and T47D cells was experimentally manipulated as described in the legend to FIG. 11 and the invasiveness of the cells measured in Boyden Chamber assays. 13b shows crystal violet stained Boyden Chamber inserts for PAPPA depleted (KD) BT549 cells and PAPPA overexpressing (PAPPA+) T47D cells compared to untreated (UT) and control-transfected (CO) cells. 13c shows surface β1-integrin levels in CO and KD BT549 cells. 13d shows the increased invasiveness associated with PAPPA depletion in BT549 cells was reversed by addition of an anti-β1-integrin blocking antibody to the culture medium for the duration of the invasion assay;

FIG. 14 shows cell growth characteristics and PAPPA expression in T47D cells. (A) is a Brightfield image of T47D cells (20× objective). The population doubling time was 44 hours which was calculated using a Countess™ automated cell counter (Life technologies) (B) shows the DNA content of untreated T47D cells following PI staining. The data shown were analysed using Mulitcycle AV software (C) Quantitative PCR was used to measure the relative levels of mRNA encoding PAPPA in T47D and BT549 cells. Primers spanning exons 14-15 were used to detect PAPPA and exons 4-5 to detect the endogenous control RPLP0 (D) The PCR products from the experiment described in (C) were subjected to agarose gel electrophoresis (E) shows a Western blot of T47D and BT549 cytosolic fractions probed with rabbit polyclonal anti-PAPP-A1 antibody. β-actin loading controls indicated in the lower panel (F) MethyLight assays were performed in genomic DNA samples isolated from T47D and BT549 cells. PMR indicates the percentage methylated reference which is obtained by dividing the PAPPA:COL2A1 ratio of a sample by the PAPPA:COL2A1 ratio of the SssI-treated human white blood cell DNA and multiplied by 100; and

FIG. 15 shows Exogenous IGF-1 reverses the mitotic delay phenotype observed inT47D cells. (A) shows representative images (original magnification, 200×) of control and IGF-1 treated T47D cells immuno-stained with H3S10ph antibody. Indicated by arrows are the mitotic cells in prophase/prometaphase. (B) shows the percentage of total mitotic cells in prophase/prometaphase in T47D control and IGF-1 treated cells.

### DESCRIPTION OF THE INVENTION

The term “patient” refers to any animal (e.g. mammal), including, but not limited to, humans, non-human primates, canines, felines, rodents and the like, which is to be the recipient of the diagnosis. Typically, the term “patient” is used herein in reference to a human subject.

The terms “cancer” and “cancerous” refer to or describe the physiological condition in mammals in which a population of cells are characterised by unregulated cell growth.

The terms “cancer cell” and “tumour cell” are grammatical equivalents referring to the total population of cells derived from a tumour or a pre-cancerous lesion.

The term “breast cancer” includes all forms of primary breast carcinoma, including invasive ductal carcinoma, invasive lobular carcinoma, tubular carcinoma, medullary carcinoma, alveolar carcinoma, solid variant carcinoma, signet ring cell carcinoma, metaplastic carcinoma.

The term “invasive cancer” refers to cancer that has spread beyond the primary tumour in which it developed, and is growing in surrounding, healthy tissues. Invasive cancer is sometimes referred to as infiltrating cancer. The term is intended to include all primary invasive breast cancers including, invasive ductal carcinoma “not otherwise specified” (IDC) and IDC subtypes (e.g. mixed, pleomorphic, osteoclast types), invasive lobular carcinoma (ILC), tubular carcinoma, mucinous carcinoma, medullary carcinoma, neuroendocrine tumours, invasive papillary and cribriform carcinoma and invasive apocrine, metaplastic and oncocytic subtypes.

As used herein, the phrase “risk of invasive cancer” refers to the risk of progression from in-situ non-invasive cancer to invasive cancer. Risk of invasive cancer can also refer to a risk of recurrent in-situ non-invasive cancer.

As used herein, the phrase “risk of recurrent disease” refers to the risk of in situ non-invasive proliferative lesions occurring at a different location within the breast tissue of the patient.

Preferably the tissue sample obtained from the patient and used in the in vitro methods of the invention is a breast tissue sample that exhibits proliferative lesions. As used herein, the term “proliferative lesions” refers to lesions with atypia. Proliferative lesions with atypia represent precursor lesions of invasive breast cancer. Precursor lesions can be divided broadly into two groups, namely “pre-invasive lesions” and “proliferative lesions with uncertain malignant potential”. These two groups of entities represent proliferation of atypical or malignant cells within the breast parenchymal structures but in which there is no evidence of invasion across the basement membrane. Pre-invasive lesions include ductal carcinoma-in-situ (DCIS), lobular carcinoma-in-situ (LCIS) and Paget's disease of the nipple. Proliferative lesions with uncertain malignant potential include such entities as lobular neoplasia, lobular intraepithelial neoplasia, atypical lobular hyperplasia (ALH), flat epithelial atypia (FEA), atypical ductal hyperplasia (ADH) microinvasive carcinoma, intraductal papillary neoplasms and phyllodes tumour. These entities are well characterised in the art and used in routine clinical pathological practice.

The methods of the invention described herein are carried out in vitro. For the avoidance of doubt, the term “in vitro” has its usual meaning in the art, referring to methods that are carried out in or on tissue in an artificial environment outside the body of the patient from whom the tissue sample has been obtained.

The terms “immunoassay”, “immuno-detection” and “immunological assay” are used interchangeably herein and refer to antibody-based techniques for identifying the presence of or levels of a protein in a sample.

The term “antibody” refers to an immunoglobulin which specifically recognises an epitope on a target as determined by the binding characteristics of the immunoglobulin variable domains of the heavy and light chains (V_{H}S and V_{L}S), more specifically the complementarity-determining regions (CDRs). Many potential antibody forms are known in the art, which may include, but are not limited to, a plurality of intact monoclonal antibodies or polyclonal mixtures comprising intact monoclonal antibodies, antibody fragments (for example F_{ab}, F_{ab}′, and Fᵣ fragments, linear antibodies, single chain antibodies, and multispecific antibodies comprising antibody fragments), single chain variable fragments (scFᵥS), multispecific antibodies, chimeric antibodies, humanised antibodies and fusion proteins comprising the domains necessary for the recognition of a given epitope on a target. Antibodies may also be conjugated to various moieties for a diagnostic effect, including but not limited to radionuclides, fluorophores or dyes.

The term “specifically recognises”, in the context of antibody-epitope interactions, refers to an interaction wherein the antibody and epitope associate more frequently or rapidly, or with greater duration or affinity, or with any combination of the above, than when either antibody or epitope is substituted for an alternative substance, for example an unrelated protein. Generally, but not necessarily, reference to binding means specific recognition.

The term “mitosis” has its usual meaning in the art. Mitosis is the process by which a eukaryotic cell separates the chromosomes in its cell nucleus into two identical sets, in two separate nuclei. Mitosis and cytokinesis together define the mitotic (M) phase of the cell cycle. The process of mitosis is characterised into stages corresponding to the completion of one set of activities and the start of the next. These stages are prophase, prometaphase, metaphase, anaphase and telophase.

The term “prophase” has its usual meaning in the art. Prophase refers to the stage where the chromatin in the nucleus becomes tightly coiled, condensing into discrete chromosomes.

The term “prometaphase” has its usual meaning in the art. During prometaphase the nuclear membrane disintegrates and microtubules invade the nuclear space.

The present invention has identified that suppression of endogenous PAPPA levels is implicated in the development of breast cancer. More particularly, the present invention has identified that suppression of PAPPA is implicated in the “invasiveness” of breast cancer. This is a significant breakthrough in breast cancer detection and treatment as it allows “at risk” patients to be identified and therapies to be developed in a targeted way.

This invention enables methods to be developed for determining the risk of breast cancer in a patient based on detecting the presence/absence of the Pregnancy-Associated Plasma Protein A (PAPPA) or the methylation state of the gene or its promoter sequence, or the loss of function of PAPPA.

PAPPA was identified in 1974 as one of four proteins of placental origin circulating at high concentrations in pregnant women, and later found clinical utility as a biomarker for Down's syndrome pregnancies. Its biological function remained an enigma for a quarter of a century until it was identified as a protease that regulates IGF bioavailability through cleavage of the inhibitory insulin-like growth factor binding protein-4 (IGFBP-4). Its role as an IGFBP-4 protease in a diverse range of cell types (e.g. fibroblasts, osteoblasts and vascular smooth muscle cells), together with a highly conserved amino acid sequence in vertebrates, indicated that PAPPA serves a basic function beyond placental physiology. The inventors have now shown that in breast tissue PAPPA is required for normal progression through mitosis. The endogenous suppression of PAPPA causes delay or arrest in early stages of mitosis, with cycling cells stalling in prophase/prometaphase. This can be reversed by increasing the expression of the endogenous PAPPA gene or by introducing artificial constructs which express PAPPA.

Mitotic delay due to PAPPA suppression in breast cancer cells at first glance appears disadvantageous to tumour growth. However a major biological advantage is conferred to the mitotically delayed, neoplastic breast cell through the associated increase in acquiring invasive capacity. In breast cancer specimens, mitotic delay linked to PAPPA silencing can be detected in virtually all cases of invasive cancer and also in a proportion of non-invasive lesions. The gain in invasive capacity as a consequence of PAPPA loss therefore occurs early in multi-step mammary tumour progression during the transition from non-invasive to invasive cancer. Detection of PAPPA deregulation and mitotic delay in clinical biopsy specimens offers a significant advance in identifying breast cancer patients with pre-invasive lesions, such as ductal carcinoma-in-situ, atypical hyperplasia or non-invasive lobular carcinoma in situ, who are at higher risk of developing invasive disease. Accordingly, the present invention can be used to discriminate patients exhibiting pre-invasive lesions into those whose lesions are unlikely to progress to an invasive phenotype (and who may not require additional therapy) and those predisposed to the invasive phenotype (and who may therefore require additional therapy).

The inventors have identified that one cause of PAPPA suppression in breast cancer cells (or pre-cancerous cells) is due to methylation of its DNA, primarily the PAPPA promoter region. DNA methylation, caused primarily by covalent addition of methyl groups to cytosine within CpG dinucleotides, occurs primarily in promoter regions of genes due to the large proportion of CpG islands found there. Hypermethylation results in transcriptional silencing.

Detecting the presence or absence of cancer by determining the methylation state of specific genes is known (but not in the context of PAPPA), and conventional methods for doing this may be adapted for use in the present invention. For example, methylation-specific PCR (MSP) has been used to determine the methylation status of specific genes. This technique, referred to also as MethyLight is described in Eads et al, Nucleic Acids Res. 2000; 28(8), and Widschwendler et al, Cancer Res., 2004; 64:3807-3813, the content of each of which is incorporated herein by reference. Alternative methods include Combined Bisulphate Restriction Analyses, Methylation-sensitive Single Nucleotide Primer Extension and the use of CpG island microarrays. Commercially available kits for the study of DNA methylation are available. Accordingly, the present invention makes use of conventional methods for determining the methylation state of the PAPPA gene or its regulatory promoter sequences, for the determination of breast cancer or the risk of progressing to invasive breast cancer in a patient.

MethyLight is a high-throughput quantitative methylation assay that utilises fluorescence-based real-time PCR (TaqMan®) technology that requires no further manipulations after the PCR step. MethyLight is a highly sensitive assay, capable of detecting methylated alleles in the presence of a 10,000-fold excess of unmethylated alleles. The assay is also highly quantitative and can very accurately determine the relative prevalence of a particular pattern of DNA methylation using very small amounts of template DNA.

According to the present invention, MethyLight can be used to determine the methylation state of the PAPPA gene or regulatory or promoter regions in a sample of genomic DNA obtained for the patient's sample. Determination of the methylation state of the PAPPA gene may comprise the following steps:
i. Genomic DNA is extracted from the breast tissue sample and treated with sodium bisulfite to convert unmethylated cytosines to uracil residues (methylated residues are protected);
ii. Primers and probes designed specifically for bisulfite-modified DNA, such as those detailed in Table 2 are used to amplify the bisulfite-targeted DNA sample. The primer/probe sets used include a methylated set specific for the PAPPA gene (for example, see SEQ ID Nos. 7-9 in Table 2) and a set specific for a reference gene (COL2A1) (for example, see SEQ ID Nos. 31-33 in Table 2);
iii. The data is analysed and Ct values are calculated, for example by using ABI Step one plus software;
iv. The percentage of fully methylated PAPPA molecules at the specific locus is calculated by dividing the PAPPA:COL2A1 ratio of a sample by the PAPPA:COL2A1 ratio of a positive control sample (for example, SssI treated HeLa genomic DNA) and multiplying by 100.

Since MethyLight reactions are specific to bisulfite converted DNA, the generation of false positive results is precluded.

Although DNA methylation (hypermethylation) is one cause of PAPPA suppression, there may be other causes. For example, the PAPPA gene (or its regulatory sequences) may be mutated leading to transcriptional silencing. Point mutations, deletions, loss of heterozygosity, translocations etc. may all cause the PAPPA gene to lose transcriptional activity. While modification at the genetic level may cause reduced (or no) expression of PAPPA, it may also be that modification (mutation) at the genetic level results in expression of PAPPA with reduced or no functional activity. Accordingly, the present invention envisages that PAPPA activity levels be used to help make a diagnosis. Mutation hot spots may be identified which contribute to the loss of activity and identifying such hot spots in a patient sample can also contribute to the diagnosis.

Loss of heterozygosity can be measured using various techniques, including semi quantitative RT-PCR analysis. PAPPA is localised to human chromosome 9q32-33.1. Total RNA can be extracted using commercially available RNA extraction kits and reverse transcription can be performed using a reverse transcriptase enzyme. Unique primers can be designed within the PAPPA gene region and RT-PCR reactions can be performed in a thermal cycler. Levels of expression of PAPPA gene can be determined by the ratio of the band intensity of PAPP-A gene compared to an endogenous control.

Real-time PCR reactions can also be performed to quantitatively confirm the results obtained from RT-PCR as will be appreciated by the skilled person. Unique primers can be designed for PAPPA and an endogenous control. Real-time PCR can be carried out to generate a standard curve for each gene under investigation. The fold reduction of PAPPA can be normalized to that of an endogenous control to compensate for the amount of RNA in each sample and also to account for the differences in the efficiency of the reverse transcription reaction.

Other methods used for the detection of loss of heterozygosity are high-resolution PCR based fluorescence quantitation using capillary electrophoresis systems, amplification of microsatellites by PCR using radiolabelled nucleotides followed by autoradiography and next generation sequencing (Ion Torrent™, Life Technologies).

As mentioned previously, point mutations may be responsible for PAPPA loss or PAPPA loss of functional activity. There are a variety of methods available for the detection of point mutations in molecular diagnostics. The choice of the method to be used depends on the specimen being analysed, how reliable the method is, whether the mutations to be detected are known before analysis and the ratio between wild-type and mutant alleles.

Denaturing gradient gel electrophoresis is a further technique for mutation detection, particularly for point mutations. A prolonged (48 hr) proteinase K digestion method or DNA easy kit (Qiagen) can be used to extract genomic DNA. Double stranded DNA (PCR fragments of 1 kb) can be generated by multiplex PCR reaction covering the whole of the PAPPA coding region. In order to increase the efficiency of detection GC clamps can be attached to one of the PCR primers. The DNA can then be subjected to increasing concentrations of a denaturing agent like urea or formamide in a gel electrophoresis set up. With increasing concentrations of denaturing agent domains in the DNA will dissociate according to their melting temperature (Tm). DNA hybrids of 1 kb usually contain about 3-4 domains, each of which would melt at a distinct temperature. Dissociation of strands in such domains results in the decrease of electrophoretic mobility, and a 1 bp difference is sufficient to change the Tm. Base mismatches in the heteroduplices lead to a significant destabilisation of domains resulting in differences in Tm between homoduplex and heteroduplex molecules. The homo and heteroduplices will be detected by silver staining after gel electrophoresis. This method offers the advantage that 100% of point mutations can be detected when heteroduplices are generated from sense and antisense strands (Cotton RG, Current methods of mutation detection, Mutat Res 1993; 285: 125-44).

Alternative methods available for the detection of point mutations include PCR-single stranded conformation polymorphism, heteroduplex analysis, protein truncation test, RNASE A cleavage method, chemical/enzyme mismatch cleavage, allele specific oligonucleotide hybridisation on DNA chips, allele specific PCR with a blocking reagent (to suppress amplification of wild-type allele) followed by real time PCR, direct sequencing of PCR products, pyrosequencing and next generation sequencing systems.

As mentioned previously, PAPPA loss and/or PAPPA loss of functional activity may be due to insertions, deletions and frame-shift mutations. The technique of pyrosequencing can be used for detection of insertions, deletions, frame-shift mutations. Pyrosequencing is based on the sequencing-by-synthesis principle. In this method a single-stranded PCR/RT-PCR fragment is used as a template for the reaction. During the process of DNA replication after nucleotide incorporation, released PPi (inorganic phosphate) is converted to light by an enzymatic cascade; ATP sulfurylase which converts PPi to ATP in the presence of APS. This ATP would further drive the luciferase mediated conversion of luciferin to oxyluciferin that generates visible light, which can be detected by a CCD sensor and is visible as a peak in the pyrogram (Ronaghi, M., Uhlen, M., and Nyren, P, A sequencing method based on real-time pyrophosphate, Science; 1998b 281: 363-365). The light signal generated is linearly proportional to the nucleotides incorporated.

A prolonged (48 hr) proteinase K digestion method or DNA easy kit (Qiagen) can be used to extract genomic DNA from the patient tissue sample. PCR and sequencing primers for the PAPP-A gene can be designed for use in pyrosequencing. PCR products can be bound to streptavidin-sepharose, purified washed and denatured using NaoH solution and washed again. Then the pyrosequencing primer can be annealed to the single-stranded PCR product and the reaction carried out on, for example, a Pyromark ID system (Qiagen) according to the manufacturer's instructions.

Other methods available for detecting insertions/deletions and frame-shift mutations are big dye terminator sequencing, next generation sequencing systems and heteroduplex analysis using capillary/microchip based electrophoresis.

Alternative methods of the invention require determining the presence (or absence) or the level of PAPPA in a patient breast tissue sample. This can be carried out by determining protein levels, or by studying the expression level of the gene coding for the protein. As used herein the term “expression level” refers to the amount of the specified protein (or mRNA coding for the protein) in the breast tissue sample. The expression level is then compared to that of a control. The control may be a tissue sample of a person that is known to not have cancer or may be a reference value. It will be apparent to the skilled person that comparing expression levels of a control and the test sample will allow a decision to be made as to whether the expression level in the test sample and control are similar or different and therefore whether the patient has or is at risk of invasive breast cancer.

Methods of measuring the level of expression of a protein from a biological sample are well known in the art and any suitable method may be used. Protein or nucleic acid from the sample may be analysed to determine the expression level, and examples of suitable methods include semi-quantitative methods such as in situ hybridisation (ISH) fluorescence and in situ hybridisation (FISH), and variants of these methods for detecting mRNA levels in tissue or cell preparations, Northern blotting, and quantitative PCR reactions. The use of Northern blotting techniques or quantitative PCR to detect gene expression levels is well known in the art. Kits for quantitative PCR-based gene expression analysis are commercially available, for example the Quantitect system manufactured by Qiagen. Simultaneous analysis of expression levels in multiple samples using a hybridisation-based nucleic acid array system is well known in the art and is also within the scope of the invention. Mutation-specific PCR may also be used, as will be appreciated by the skilled person.

PAPPA levels in a breast tissue sample can be determined using conventional immunological detection techniques, using conventional anti-PAPPA antibodies. The antibody having specificity for PAPPA, or a secondary antibody that binds to such an antibody, can be detectably-labelled. Suitable labels include, without limitation, radionuclides (e.g. ¹²⁵I, ¹³¹I, ³⁵S, ³H, ³²P or ¹⁴C), fluorophores (e.g. Fluorescein, FITC or rhodamine), luminescent moieties (e.g. Qdot nanoparticles supplied by Quantum Dot Corporation, Palo Alto Calif.) or enzymes (e.g. alkaline phosphatase or horse radish peroxidase).

Immunological assays for detecting PAPPA can be performed in a variety of assay formats, including sandwich assays e.g. (ELISA), competition assays (competitive RIA), bridge immunoassays, immunohistochemistry (IHC) and immunocytochemistry (ICC). Methods for detecting PAPPA include contacting a patient sample with an antibody that binds to PAPPA and detecting binding. An antibody having specificity for PAPPA can be immobilised on a support material using conventional methods. Binding of PAPPA to the antibody on the support can be detected using surface plasmon resonance (Biacore Int, Sweden). Anti-PAPPA antibodies are available commercially (e.g. HPA001667 from Sigma-Aldrich, MA1-46425 (5H9) from Thermo Scientific, OASAO3208 from Aviva Systems Biology and AO230 from Dako). The immuno-detection of PAPPA is also disclosed in U.S. Pat. No. 6,172,198, the content of which incorporated herein by reference.

In order to enable immunohistochemical detection of PAPPA within a breast tissue sample, formalin-fixed, paraffin-embedded breast tissue sections are prepared and mounted on SuperFrost++ charged slides. Following epitope retrieval by proteolytic digestion, endogenous peroxidase activity is quenched and the sections are incubated with a first anti-PAPPA antibody (available, for example, from DAKO). The section is then further incubated with a polymer-linked secondary antibody and peroxidase which enables a chromogenic signal to develop following addition with DAB, thereby allowing binding of the first antibody to the PAPPA protein to be detected visually. The immunohistochemical procedure described above can be fully automated using commercially available immunostainers.

PAPPA protein expression can be classified using conventional methods, for example, membrane and cytoplasmic staining intensity can be evaluated using the following scoring system: negative (0), no staining is observed; weakly positive (1+), a faint/barely perceptible membrane/cytoplasmic staining is detected in more than 25% of cells; moderately positive (2+), weak staining is detected in more than 25% of cells; strongly positive (3+), strong membrane/cytoplasmic staining is detected in more than 25% of cells. Any focal staining of less than 25% of tumour cells is considered as 1+.

For analysis of a relatively small number of PAPPA proteins, a quantitative immunoassay such as a Western blot or ELISA can be used to detect the amount of protein (and therefore level of expression) in a breast tissue sample. Semi-quantitative methods such as IHC and ICC can also be used.

To analyse a larger number of samples simultaneously, a protein array may be used. Protein arrays are well known in the art and function in a similar way to nucleic acid arrays, primarily using known immobilised proteins (probes) to “capture” a protein of interest. A protein array contains a plurality of immobilised probe proteins. The array contains probe proteins with affinity for PAPPA.

Alternatively, 2D Gel Electrophoresis can be used to analyse simultaneously the expression level of PAPPA. This method is well known in the art; a sample containing a large number of proteins are typically separated in a first dimension by isoelectric focusing and in a second dimension by size. Each protein resides at a unique location (a “spot”) on the resulting gel. The amount of protein in each spot, and therefore the level of expression, can be determined using a number of techniques. An example of a suitable technique is silver-staining the gel followed by scanning with a Bio-rad FX scanner and computer aided analysis using MELANIE 3.0 software (GeneBio). Alternatively, Difference Gel Electrophoresis (DIGE) may be used to quantify the expression level (see Von Eggeling et al; Int. J. Mol Med. 2001 October; 8(4):373-7.

Typically, there is a risk of progression to invasive breast cancer and/or risk of recurrent non-invasive disease if PAPPA is not present or is present at a level less than 90%, 80%, 70%, 60%, 50%, 40%, 30% or 20% compared to the control. Most typically, if the patient is at risk of progressing to invasive breast cancer and/or at risk of recurrent non-invasive disease, PAPPA will be present at an amount between 40%-80% of that of the control. Most typically, PAPPA will be present at a level between 50%-70%, e.g. approx. 60% compared to that of a control. The control can be a patient sample from normal breast tissue, or may be a reference value.

The method of the first aspect of the present invention will be carried out typically to establish whether PAPPA is present in the tissue sample at reduced levels compared to a control. It is also envisaged that PAPPA protein may be present at or near to normal levels, but the expressed protein is inactive, or active at reduced levels. Accordingly, the invention encompasses monitoring the activity of PAPPA. In this context, the reference to whether PAPPA is present (or is present at a reduced level) compared to a control, encompasses the functional activity of PAPPA.

PAPPA activity can be measured using conventional techniques. For example, PAPPA activity can be determined by examining IGFBP-4 proteolytic activity in a sample. Methods for detecting PAPPA activity are disclosed in US patent publication No. 2005/0272034, the content of which is incorporated herein by reference. Alternatively, loss of PAPPA activity may also be determined by mutation-specific PCR analysis.

In one embodiment, PAPPA activity may be detected by screening for proteolytic cleavage of its substrate IGFBP-4 using immunoblotting. PAPPA secreted into the medium can be detected by incubating the media samples in a buffer, such as 50 mM Tris (pH 7.5) supplemented with IGFBP-4. Samples can then be incubated (for example, at 37° C. for 4 hrs) and the proteolytic products detected by immunoblotting using available commercial antibodies against IGBP4 protein.

Alternatively, PAPPA activity can be detected by using an ELISA (Enzyme linked immunosorbent assay), wherein specific antibodies against PAPPA are immobilised in the well of a microtitre plate. After washing away unbound protein the activity of PAPPA can be measured using a synthetic substrate which liberates a coloured product only if the primary specific reaction between PAPPA and its antibody has occurred and the bound PAPPA is active. The colour developed is quantified spectrophotometrically using a microplate reader.

Alternatively, the interaction between PAPPA and its substrate IGFBP-4 can also be assessed using Biacore (Surface plasmon resonance technology) or Fluorescence polarisation assay. These methods offer the advantage of being very sensitive and specific and can easily be adapted to develop a high-throughput assay.

As a control for the above described methods a mutant PAPP-A protein (E483Q) which is proteolytically inactive may be used.

PAPPA activity may be reduced by greater than 20%, 30%, 40%, 50%, 60%, 70%, 80%, 90% or greater than 90% compared to a control, in a tissue sample from a patient at risk of invasive breast cancer.

Another aspect of the invention is directed to determining whether cells in a breast tissue sample are stalled in mitosis. The present invention provides an in vitro method for determining whether breast cells are stalled in mitosis, by identifying a delayed mitotic phenotype. Identification of this phenotype comprises identifying the proportion of mitotic cells in a tissue sample obtained from a patient that are in prophase or prometaphase and comparing to a pre-determined cut-off value.

The pre-determined cut-off value is at least 30% and preferably at least 33%. Therefore, if at least 30% of mitotic cells in the tissue sample are identified as being in prophase or pro-metaphase then the delayed mitotic phenotype is identified in the tissue sample. The pre-determined cut-off value may also be set higher than this, for example, at least 35%, 40%, 50%, 60%, 70% or more.

In order for the analysis to be statistically significant, at least five of the cells within the tissue sample must be undergoing mitosis. If at least 30% of these at least five mitotic cells are identified as being in prophase or prometaphase then the tissue sample is identified as having a delayed mitotic phenotype.

In another related aspect of the invention, a diagnosis to determine the risk of progression of a proliferative lesion to invasive breast cancer and/or the risk of recurrent non-invasive disease can be made by identifying the proportion of mitotic cells in a breast tissue sample obtained from a patient that are in prophase or pro-metaphase, and comparing to a pre-determined cut-off value.

The pre-determined cut-off value is at least 30% and preferably at least 33%. Therefore, if at least 30% of mitotic cells in the tissue sample are identified as being in prophase or pro-metaphase then there is a risk of progression to invasive cancer and/or risk of recurrent non-invasive disease.

In order for the analysis to be statistically significant, at least five of the cells within the tissue sample must be undergoing mitosis. If at least 30% of these at least five mitotic cells are identified as being in prophase or prometaphase then the tissue sample is deemed to have a delayed mitotic phenotype.

According to an aspect of the invention, if a delayed mitotic phenotype is identified, this indicates that there is the risk of the proliferative lesion progressing to invasive breast cancer and/or the risk of recurrent non-invasive disease. For example, if five of the cells within the tissue sample are in identified as being in mitosis, at least two of these cells must be in prophase/prometaphase in order for the mitotic delay phenotype to be identified and/or for risk of the proliferative lesion progressing to invasive cancer and/or risk of recurrent non-invasive disease to be determined. More preferably the proportion of mitotic cells in prophase/prometaphase in a breast tissue sample from a patient having the delayed mitotic phenotype is greater than 33%, 35%, 40%, 45%, 50%, 55%, 60%, 65%. Typically, a “control” value for non-invasive healthy breast tissue cells undergoing mitosis would be approximately 10-25%, e.g. 23% cells in prophase/prometaphase.

If fewer than five cells in a tissue sample are undergoing mitosis then the analysis of the proportion of mitotic cells that are in prophase/prometaphase will not be sufficiently significant to enable the delayed mitotic phenotype to be identified according to the methods of the invention. Therefore, the methods require there to be at least five mitotic cells in the tissue sample being analysed at the time of analysis.

Detection of whether the cells of the tissue sample are in prophase or pro-metaphase can be carried out using techniques conventional in the art. For example, immuno-detection techniques using specific antibodies are often used to characterise the mitotic phase of a cell. Immunohistochemistry (IHC) is an immuno-detection technique and refers to the process of detecting antigens in cells of a tissue section by visualising an antibody-antigen interaction. This can be achieved by tagging an antibody with a reporter moiety, preferably a visual reporter such as a fluorophore (termed “immunofluorescence”) or by conjugating an antibody to an enzyme, such as peroxidase, that can catalyse a colour-producing reaction that can be detected and observed.

H3S10 phosphorylation (H3S10ph) is a mitosis-specific modification essential for the onset of mitosis; the phosphorylation of the serine 10 at Histone H3 is important for chromosome condensation. Antibodies specific for H3S10ph are commercially available (e.g. Millipore and Active Motif) as are kits for carrying out mitotic assays.

Therefore, according to a further aspect of the invention, immuno-detection is used to determine the risk of proliferative lesions progressing to invasive breast cancer and/or the risk of recurrent non-invasive disease. Preferably, immuno-detection is carried out using an H3S10ph antibody.

Alternative markers of mitosis are also available commercially and may be utilised in the methods of the invention. The characterisation of the different phases of mitosis is well known in the art as will be appreciated by the skilled person.

The analysis can be carried out to provide a “snapshot” of the different phases of mitosis for a tissue sample. In this way, a mitotic phase distribution analysis is obtained which is then used to characterise the proportion of mitotic cells that are in prophase or prometaphase.

In order to enable immunohistochemical detection of a mitotic marker (such as H3S10ph), formalin-fixed, paraffin-embedded breast tissue sections are prepared and mounted on SuperFrost++ charged slides. Following heat-mediated epitope retrieval, endogenous peroxidase activity is quenched and the sections are incubated with a first antibody (suitable H3S10ph antibodies are available, for example, from Millipore) which specifically recognises mitotic markers (such as phosphorylated H3S10) within mitotic cells. The section is then further incubated with a polymer-linked secondary antibody and peroxidase which enables a chromogenic signal to develop following addition with DAB, thereby allowing binding of the first antibody to the mitotic marker to be detected visually. The immunohistochemical procedure described above can be fully automated using commercially available immunostainers.

In order to analyse mitotic phase distribution in a breast tissue sample or other patient sample (e.g. nipple aspirate) or cultured cell line, at least two consecutive serial sections from each sample and at least two cytospin preparations for each cell line or body fluid (e.g. aspirate) are immunolabelled as described above and five to twenty high power fields (400× magnification) are image captured and a minimum of 5 mitotic cells for each sample are used to determine the mitotic phase distribution. All mitotic cells within the captured fields can be classified based on their chromosomal morphology as prophase/prometaphase, metaphase, anaphase and telophase, according to classical morphological criteria. A population of cells is classified as ‘delayed’ if at least 30% of mitotic cells reside in prophase/prometaphase.

The breast tissue sample analysed by any of the methods described herein will be taken from a patient exhibiting proliferative lesions. The tissue sample may include pre-invasive lesions, including ductal carcinoma-in-situ (DCIS), lobular carcinoma-in-situ (LCIS) and Paget's disease of the nipple, and proliferative lesions with uncertain malignant potential, including such entities as lobular neoplasia, lobular intraepithelial neoplasia, atypical lobular hyperplasia (ALH), flat epithelial atypia (FEA), atypical ductal hyperplasia (ADH) microinvasive carcinoma, intraductal papillary neoplasms and phyllodes tumour. Most preferably, the tissue sample will exhibit DCIS. Methods for taking a sample from a patient (biopsy) will be apparent to the skilled person.

In addition to the diagnostic methods of the invention, the present invention provides molecules able to replace or increase PAPPA activity levels/function in a cell, for use in the treatment of breast cancer. For example, PAPPA protein, or nucleic acid able to express PAPPA, or an agonist of the IGF receptor (e.g. IGF-1) may be used to counteract the effect of PAPPA suppression in a patient. Methods for the delivery of proteins or nucleic acids to sites in an organism are well known and may be used in the present invention.

As methylation of DNA is an epigenetic modification, and can be reversed to allow the cells to express PAPPA and progress through a normal mitosis, demethylating drugs are an attractive therapeutic option for promoting mitotic cell division. Therefore, where suppression is caused by hypermethylation of PAPPA DNA, the therapeutic can be:
i. a molecule able to reverse the methylation of the PAPPA gene or regulatory or promoter sequences;
ii. an agent comprising a moiety that competitively binds to methyl groups and/or prevents methylation at cytosines (i.e. an inhibitor of DNA methylation);
iii. an antagonist/inhibitor of DNA methyl transferase (DMT); or
iv. antisense oligonucleotides against the region of the PAPPA gene promoter comprising a CpG island.

One or more or all of these agents that relate to and/or affect methylation or demethylation at CpG sites on the PAPPA promoter may be used as a therapeutic according to the present invention. Antagonists or inhibitors can be any molecule capable of antagonising or inhibiting the target bio-activity. Therefore, antagonists or inhibitors can be, for example, small molecules, proteins, polypeptides, peptides, oligonucleotides, lipids, carbohydrates, polymers and the like.

Suitable demethylation drugs include decitabine (5-aza-2′-deoxycytidine), farazabine, azaytidine (5-azacytidine), histone deacetylase inhibitors (such as hydroxamic acids (e.g. trichostatin A), cyclic tetrapeptides (e.g. trapoxin B), depsipeptides, benzamides, electrophilic ketones, aliphatic acid compounds (e.g. phenylbutyrate and valproic acid), hydroxamic acids (e.g. vorinostat, belinostat and panobinostat) and benzamides) and phenylbutyrates.

Targeting suitable demethylating drugs to the PAPPA gene or promoter sequences is possible and compounds to do this are within the scope of the present invention.

The demethylating agent can be delivered by any delivery method, including by systemic administration. Delivery can also be intraductally to the breast duct in a patient. Delivery to the breast duct may be accomplished, for example, using a delivery tool such as a catheter or cannula and infusing the demethylating agent in a suitable medium or solution to contact target ductal epithelial cells. The amount of the agent can vary, but will be an amount sufficient to target all atypical cells in the duct and an amount sufficient to inhibit or reverse DNA methylation on PAPPA promoters expressed in target ductal epithelial cells.

The present invention also allows conventional chemotherapeutics to be given, but increases the effectiveness of these. For example, many chemotherapeutic drugs act on cells undergoing mitosis, and specifically act on cells that are in a stage of mitosis following prophase/prometaphase. These are less effective when the cancer cell has stalled in mitosis. By administering agents able to allow the cancer cells to progress through mitosis, conventional chemotherapeutics can then act on the cells. Accordingly, the cancer cells cannot avoid the drug treatment due to mitotic inactivity. Suitable chemotherapeutics which may be administered after releasing the mitotic block include taxanes (taxol) and vinca alkaloids (e.g. vinblastine, vincristine, vindesine, and vinorelbine).

A further aspect of the invention provides a therapeutic regimen (or method) for treating or preventing breast cancer in a patient. The regimen comprises the sequential administration of two drugs: a first drug that releases mitotically delayed cancer cells and promotes unperturbed transit through prophase, prometaphase, metaphase, anaphase, telophase and cytokinesis; and a second drug which is a chemotherapeutic agent, preferably a chemotherapeutic agent that targets mitosis after prophase/prometaphase.

This aspect of the invention is based upon the observation that mitotically-dividing breast cells that become stalled in prophase/prometaphase (termed herein “mitotic block”) are not sensitive to chemotherapeutic agents that are active against a dividing cell at a stage in mitosis after prophase/prometaphase. Therefore, by first administering to a patient a drug which releases the mitotic cells from the mitotic block, the cells are able to progress through the normal mitotic cycle (i.e. from prophase/prometaphase to metaphase, anaphase, telophase (and cytokinesis)), thereby becoming sensitive to subsequently/sequentially administered chemotherapeutic agents.

The first drug may be PAPPA protein, or a nucleic acid encoding functional PAPPA, an IGF receptor agonist (e.g. IGF-1) or a demethylation agent. The second drug may be any drug affecting proliferating cells, and is preferably an anti-mitotic chemotherapeutic agent that targets mitosis after prometaphase. Preferably the second drug is selected from the group comprising taxanes and vinca alkaloids.

The term “sequentially” is understood to mean that the first and second drugs must exert their respective biological effects in that specific order. The effect of administering the first drug is that the mitotic block is released, thereby enabling mitosis to progress beyond prometaphase and opening a window of opportunity for the second drug to target mitosis following prophase/prometaphase. The first and second drugs may be administered simultaneously or sequentially, provided that the first drug takes effect prior to the second drug. For example, if given together, the second drug may be in a delayed release form, such that it is active only after the mitotic block has been released.

Prior to administering the first and second drugs, it is preferable to determine whether a patient is likely to be responsive to treatment according to the therapeutic regimen of the invention.

In one embodiment, suitable patient candidates for treatment according to the therapeutic regimen of the invention are identified by determining whether mitotic cells within a breast tissue sample obtained from the patient are delayed in mitosis (i.e. determining whether the patient exhibits the delayed mitotic phenotype). Therefore, according to one embodiment, the therapeutic regimen comprises an initial step of identifying the mitotic phenotype in the patient by: (i) identifying the proportion of mitotic cells in a breast tissue sample obtained from the patient that are in prophase or pro-metaphase; and (ii) comparing to a pre-determined cut-off value. The mitotic delay phenotype is identified if the proportion of cells in prophase or pro-metaphase is greater than a cut-off value. The cut-off value is preferably at least 30% of mitotic cells within the breast tissue sample, more preferably at least 33% as described earlier. The tissue sample must contain at least five mitotic cells for the result to be statistically relevant, again as described earlier.

In another embodiment, the therapeutic regimen comprises an initial step of identifying whether the patient is a suitable candidate for treatment according to the therapeutic regimen by detecting the PAPPA loss or loss of function-related genetic alterations in the PAPPA gene, or its regulatory or promoter sequences, in a breast tissue sample obtained from the patient. If genetic alterations are present, the patient is identified as being a suitable candidate for treatment according to the therapeutic regimen of the invention. Preferably, PAPPA loss or the loss of function-related genetic alteration in the PAPPA gene or its regulatory or promoter sequences is due to methylation.

In a preferred embodiment the patient is identified as being at risk of invasive breast cancer according to the methods of the present invention and is then treated using one or more of the therapeutic applications or regimens described herein.

The present invention also envisages treatment to reduce invasiveness of breast cancer. As described above, cancer cells stalled in mitosis due to PAPPA suppression can acquire invasive capacity. By releasing the mitotic block, the cells have reduced invasive capacity. This will provide a therapeutic benefit to the patient.

In still another approach, expression of the gene encoding endogenous PAPPA can be up-regulated using suitable expression techniques. Known techniques involve the use of genetic constructs which replace the endogenous gene with an artificial alternative. Alternatively, promoter or control sequences may be inserted upstream of the endogenous gene. This may be carried out using conventional methods.

The present invention also provides breast cell lines comprising a methylated PAPPA gene promoter, for use in a screening method to detect compounds that up-regulate PAPPA. The cells may be breast cancer cells or non-invasive abnormal breast cells. The screening method can involve contacting the cell with a potential therapeutic agent and determining whether the agent up-regulates PAPPA in the cell. The agent may, for example, be a demethylating agent, or may be a nucleic acid construct which expresses PAPPA within the cell.

The present invention also provides an isolated genetic construct, for use in the treatment of breast cancer, wherein the construct comprises functional PAPPA-expressing nucleic acid, linked operably to regulatory sequences. Such constructs can be prepared using conventional technologies, as will be appreciated by the skilled person. As the PAPPA gene and promoter sequence are known, it will be readily apparent to the skilled person how to prepare a suitable construct. The PAPPA mRNA sequence is identified in NCBI accession number NM_{—}002581.3, the protein sequence is identified in NCBI accession number NP_{—}002572.2. Homologues in human or other species can be found on the NCBI database and on the MitoCheck database (www.mitocheck.org).

The therapeutics and diagnostics according to the invention are useful in the therapy and diagnosis of breast cancer.

PAPPA or other therapeutically-active agents may be formulated in combination with a suitable pharmaceutical carrier. Such formulations comprise a therapeutically effective amount of the protein (or other agent), and a pharmaceutically acceptable carrier or excipient. Such carriers include but are not limited to, saline, buffered saline, dextrose, water, glycerol, ethanol, and combinations thereof. The formulation should suit the mode of administration, and is well within the skill of the art. The invention further relates to pharmaceutical packs and kits comprising one or more containers filled with one or more of the ingredients of the compositions mentioned herein.

Proteins and other compounds of the present invention may be employed alone or in conjunction with other compounds, such as therapeutic compounds.

Preferred forms of systemic administration of the pharmaceutical compositions include injection, typically by intravenous injection. Other injection routes, such as subcutaneous, intramuscular, or intraperitoneal, can be used. Alternative means for systemic administration include transmucosal and transdermal administration using penetrants such as bile salts or fusidic acids or other detergents. In addition, if properly formulated in enteric or encapsulated formulations, oral administration may also be possible.

The dosage range required depends on the choice of protein (or other active), the route of administration, the nature of the formulation, the nature of the subject's condition, and the judgment of the attending practitioner. Suitable dosages, however, are in the range of 0.1-100 μg/kg of subject. Wide variations in the needed dosage, however, are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization, as is well understood in the art.

Proteins used in treatment can also be generated endogenously in the subject, in treatment modalities often referred to as “gene therapy” as described above. Thus, for example, cells from a subject may be engineered with a polynucleotide, such as a DNA or RNA, to encode a protein ex vivo, and for example, by the use of a retroviral plasmid vector. The cells are then introduced into the subject.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavouring agents may be provided in the pharmaceutical composition. Examples of preservatives include sodium benzoate, sorbic acid and esters of hydroxybenzoic acid. Antioxidants and suspending agents may be also used.

There may be different composition/formulation requirements dependent on the different delivery systems.

Where appropriate, the pharmaceutical compositions can be injected parenterally, for example intravenously, intramuscularly or subcutaneously. For parenteral administration, the compositions may be used in the form of a sterile aqueous solution which may contain other substances, for example enough salts or monosaccharides to make the solution isotonic with blood.

The invention is described with reference to the accompanying drawings, by the following non-limiting examples.

### Example 1

### Methods

### Tissue Specimens

Formalin-fixed, paraffin-embedded tissue was retrieved from the archives of the Department of Pathology at UCL (UCL Hospitals, London, UK) and included: invasive breast cancer (n=182, 156 of which were included for analysis of mitotic phase distribution); ductal carcinoma in situ (DCIS; n=81, 69 evaluable); normal breast tissue from reduction mammoplasty specimens (n=33, evaluability not relevant); normal breast tissue from pregnant patients (n=5, all evaluable); colon adenocarcinoma (n=41, all evaluable); transitional cell carcinoma of the bladder (n=27, all evaluable); penile squamous cell carcinoma (n=33, all evaluable); gastric adenocarcinoma (n=21, all evaluable); malignant melanoma (n=21, all evaluable); small cell lung cancer (n=30, all evaluable); and non-Hodgkin lymphoma (n=29, all evaluable). Cases were selected on the basis of available histological material and clinico-pathological information. Histological specimens had been reviewed by a qualified pathologist at diagnosis and assessed for histological subtype and nuclear grade according to the World Health Organization (WHO) criteria. A case was evaluable for mitotic phase distribution analysis if at least five mitotic cells were found on the specimen. Ethical approval was obtained from the Joint UCL/UCLH Committees on the Ethics of Human Research.

### Cytospin Preparations

To prepare cell monolayers, 0.5×10⁶ cells were cytospun onto glass slides at 800×g for 5 min using a Cytospin 4 cytocentrifuge (Thermo Scientific), air dried and fixed in 4% neutral buffered formalin overnight.

### Antibodies

PAPP-A rabbit polyclonal antibody (PAPP-A PAb) was raised against a synthetic peptide (aa1384-1399) following a 28-day immunisation protocol (Eurogentec). Other antibodies used include Histone H3 phosphorylated on serine 10 (H3S10ph) from Millipore (06-570), PAPP-A from DAKO (A0230), β-actin (AC-15) from Sigma, CD29 (HUTS-21) from BD Pharmingen, β1-integrin (MAB1959) from Chemicon and Alexa Fluor 594 from Invitrogen.

### Immunohistochemistry

Section deparaffinisation, antigen retrieval and immuno-staining were performed using the Bond III Autostainer and Bond Polymer Refine Detection kit (Leica) according to the manufacturer's instructions. Heat-mediated antigen retrieval and proteolytic digestion were used for H3S10ph and PAPPA antigens, respectively. Primary antibodies were applied for 40 min at the following dilutions: PAPP-A at 1/200; H3S10ph at 1/4000. Cytospin preparations were immuno-stained using the same protocol without the deparaffinisation step. Incubation without primary antibody was used as a negative control and sections of tonsil and placenta were used as positive controls for H3S10ph and PAPPA antibodies, respectively.

### Mitotic Phase Distribution Analysis

Two consecutive serial sections from each tissue sample and two cytospin preparations for each cell line were immuno-stained for H3S10ph for analysis of mitotic phase distribution. A minimum of 5 mitotic cells from 5-20 fields at 400× magnification were captured with a CV12 CCD camera and image capturing software (SIS). Cases were excluded from analysis if less than 5 mitotic cells were found on the specimen. Mitotic cells were classified as prophase/prometaphase, metaphase, anaphase or telophase according to conventional morphological criteria.

### Tissue Dissection

Tissue sections were deparaffinised, stained with Mayer's haematoxylin for 5 seconds, and air dried. A 100× magnification field was needle micro-dissected and genomic DNA extracted following incubation in 55 μl of 1 mg/ml proteinase K (Sigma-Aldrich) at 55° C. for 48 h.

### DNA Methylation Analysis

Genomic DNA from cell lines and cases of invasive breast cancer (n=182), DCIS (n=81) and normal breast (n=34) were used for MethyLight analysis (Widschwendter, M. et al. Cancer research (2004) 64, 3807-3813). Nine cases of invasive breast cancer, six cases of DCIS and four cases of normal breast were excluded from analysis because insufficient material for DNA extraction was found on the specimen. DNA concentration was determined by NanoDrop spectrophotometry and DNA quality was verified using qPCR with a reference gene. Mean genomic amplification of all breast samples was calculated at 34.39 cycles (SD=2.07). For all samples 400 ng of genomic DNA was bisulfite-modified using the EZ DNA Methylation-Gold Kit (Zymo Research) according to the manufacturer's instructions. Unmodified SssI treated genomic DNA (New England Biolabs) was used as positive control. Bisulfite-modified DNA was stored at −80° C. until use. Quantitative PCR analysis using MethyLight was performed for all samples. Nucleotide sequences for MethyLight primers and probes were designed in the promoter or 5′-end region of the gene of interest. Each MethyLight reaction at a specific locus covered on average 5-10 CpG dinucleotides. A detailed list of primer and probes (Metabion) for all analysed loci is provided in Tables 1 and 2. Two sets of primers and probes, designed specifically for bisulfite-modified DNA, were used for each locus; a methylated set for the gene of interest and a reference gene (COL2A1) to normalize for input DNA. Specificity of the reactions for methylated DNA was confirmed separately using SssI treated human white blood cell DNA (heavily methylated). The percentage of fully methylated molecules at a specific locus (PMR, percentage methylated reference) was calculated by dividing the GENE OF INTEREST:COL2A1 ratio of a sample by the GENE OF INTEREST:COL2A1 ratio of the SssI-treated human white blood cell DNA and multiplied by 100.

### Cell Culture

BT549, T47D, BT474, MDAMB157, MDAMB453, MCF10A and human mammary epithelial cells (HMEpC) were cultured as described in Rodriguez-Acebes et al, Am. J. Pathol, 2010; 177:2034-2045. HeLa Kyoto cells were cultured in DMEM (Invitrogen) supplemented with 10% FCS (Invitrogen) at 37° C. with 5% CO₂.

### Cell Synchronization

HeLa Kyoto cells were synchronised in S phase by a double thymidine block (Sigma). Briefly, a final concentration of 3 mM thymidine was added to the culture medium for 18 h, followed by release into fresh culture medium for 9 h and a second block with 3 mM thymidine for 17 h. HeLa Kyoto cells were synchronised in M phase by treatment with the Plk1 inhibitor BI2536 (SelleckChem) at final concentration of 5 ng/ml for 24 hours. Cell synchronisation was confirmed by flow cytometry.

TABLE 1
Location of CpG islands for MitoCheck candidate genes

| | | | CpG island | CGs in CpG | Obs CpG/ | GC |
|---|---|---|---|---|---|---|
| Gene^{a} | Gene locus | CpG island location^{b} | length (bp) | island (no.) | Exp CpG | (%) |
| PLK1 | chr16: 23,590,521-23,616,371 (16p12.1) | 23597103-23598423 | 1320 | 88 | 0.768 | 60.6 |
| TPX2 | chr20: 29,751,378-29,892,452 (20q11.2) | 29790413-29791269 | 856 | 48 | 0.823 | 55.0 |
| KIF111 | chr10: 94,327,431-94,420,670 (10q23.3) | 94341250-94341728 | 478 | 38 | 1.052 | 55.0 |
| KIF112 | chr10: 94,327,431-94,420,670 (10q23.3) | 94341912-94343451 | 1539 | 89 | 0.793 | 54.3 |
| PAPP-A | chr9: 117,893,759-118,266,552 (9q33.1) | 117955868-117957241 | 1373 | 115 | 0.852 | 64.5 |
| SGOL1 | chr3: 20,177,087-20,202,687 (3p24.3) | 20176596-20177594 | 998 | 65 | 0.876 | 55.0 |
| PSMD8 | chr19: 43,557,030-43,566,304 (19q13.2) | 43556706-43558018 | 1312 | 81 | 0.747 | 59.4 |
| TUBB2C | chr9: 139,255,532-139,257,980 (9q34) | 139254197-139256673 | 2476 | 225 | 0.817 | 67.3 |

^{a}Human Genome Organisation gene name
^{b}DNA sequences sourced using genome.ucsc,edu, CpG island locations identified using cpgislands.usc.edu

TABLE 2
Primers and probes used for MethyLight reactions

| | Forward primer | Reverse primer | |
|---|---|---|---|
| Gene^{a} | sequence 5'-3' | sequence 5'-3' | Probe sequence 5'-3' |
| KIF11 | CGAGCGTTGTATGTTGGGATT | CGCAACGAACGATAACA | 6-FAM-AACTACGCAAACATCCGCCG-BHQ-1 |
| | SEQ ID NO. 1 | TCTCA SEQ ID NO. 2 | SEQ ID NO. 3 |
| PAPPA | GCGTCGAGGTTTTTAAAGTTGGTA | CCCAACTCCAAAACCGCATAT | 6-FAM-CCCTACACCGCCACCCGAA-BHQ-1 |
| (I) | SEQ ID NO. 4 | SEQ ID NO. 5 | SEQ ID NO. 6 |
| PAPPA | GCGTGTTTGTGCGAGAGTTGT | CGCCTTCCGAATATACCCATT | 6-FAM-TCGCCCGAATATCTCTACGCCGCT- |
| (II) | SEQ ID NO. 7 | SEQ ID NO. 8 | BHQ-1 SEQ ID NO. 9 |
| PLK1 | GTTCGGGCGTTCGTGTTAAT | GCCGCGCAACACCATAA | 6-FAM-CCCTACGCAACAACAACCTTTAAACCCG- |
| (I) | SEQ ID NO. 10 | SEQ ID NO. 11 | BHQ-1 SEQ ID NO. 12 |
| PLK1 | GGTTTTATCGGCGAAAGAGATTT | CGACCCCGCACATAACG | 6-FAM-CCGACTACGTAAATCCACTAAAACCT- |
| (II) | SEQ ID NO. 13 | SEQ ID NO. 14 | BHQ-1 SEQ ID NO. 15 |
| PLK1 | CGCGTTTCGTTGTTAGATTTGAG | AAAAACCCGCGCCCTAACTA | 6-FAM-CTTCCCACGACTCACCTAACCTCG- |
| (II) | SEQ ID NO. 16 | SEQ ID NO. | BHQ-1 SEQ ID NO. 18 |
| TPX2 | TGGCGATAGGATTTTGTTGTGA | GACAACCTCCCGCAACTCTTT | 6-FAM-TTTCCTCCGTTTCCCGAACGAA-BHQ-1 |
| | SEQ ID NO. 19 | SEQ ID NO. 20 | SEQ ID NO. 21 |
| TUBB2C | AACGAACGCGCAAAATACTACA | CGTTTTGGTAGTTTATTTCGA | 6-FAM-CCGACGCTCCGCCAACGCTTA-BHQ-1 |
| | SEQ ID NO. 22 | GATTAGC SEQ ID NO 23 | SEQ ID NO. 24 |
| SGOL1 | CGCACATTCGCTCAAATCC | CGTCGAGATTCGATCGTAG | 6-FAM-CATCCGAACATCCACCTAACCCTAAAA |
| | SEQ ID NO. 25 | GTT SEQ ID NO. 26 | CG-BHQ-1 SEQ ID NO. 27 |
| PSMD8 | TTCGTCGGGTAAGCGTTTGTA | GCGACCGCCATCTTACGTAA | 6-FAM-CGGCGTTGTCGTAAATTAGGCGGTTT- |
| | SEQ ID NO. 28 | SEQ ID NO. 29 | BHQ-1 SEQ ID NO. 30 |
| COL2A1 | TCTAACAATTATAAACTCCA | GGGAAGATGGGATAGAAGGGA | 6-FAM-CCTTCATTCTAACCCAATACCTATCCCA |
| (mod) | ACCACCAA | ATAT SEQ ID NO 32 | CCTCTAAA-BHQ-1 SEQ ID NO. 33 |
| | SEQ ID NO 31 | | |
| COL2A1 | TCCGTAAGTGCAGCTTCTCTTTG | TGGAGCCCACAACTGTCAGA | 6-FAM-CAAAGTACAGAGTCAAGAGTTCCAAAG |
| (gen) | SEQ ID NO. 34 | SEQ ID NO. 35 | CCACAGA-BHQ-1 SEQ ID NO. 36 |

^{a}Human Genome Organisation gene name

### Cell Population Growth Assessment and Cell Cycle Analysis

Cell proliferation assessment and flow cytometric cell cycle analysis were performed as described in Rodriguez-Acebes et al, Am. J. Pathol, 2010; 177:2034-2045.

### RNA Interference

PAPPA was silenced by RNAi with a specific RNA duplex targeting PAPPA mRNA (Ambion s10042; sense 5′-GAGCCUACUUGGAUGUUAAtt-3′ (SEQ ID NO. 37) and antisense 5′-UUAACAUCCAAGUAGGCUCtg-3′ (SEQ ID NO. 38) or, alternatively, a pool of duplexes (ON-TARGETplus SMARTpool, Dharmacon). Non-targeting siRNA (Stealth RNAi Negative Control Med GC, Invitrogen) was used as negative control. All transfections were performed with Lipofectamine 2000 (Invitrogen). PAPPA siRNA at 100 nM final concentration was used to achieve efficient knock-down. Cells were harvested at the indicated time points post-transfection. Knock-down efficiency was assessed by qRT-PCR and/or Western blot.

### Real-Time PCR

Total RNA was isolated from cells and qRT-PCR was performed using 100 ng of total RNA as described in Tudzarova et al, EMBO J., 2010; 29:3381-3394. Primer sequences were: PAPPA forward 5′-ACAGGCTACGTGCTCCAGAT-3′ (SEQ ID NO. 39) and reverse 5′-CTCACAGGCCACCTGCTTAT-3′ SEQ ID NO. 40); RPLPO (ribosomal protein used as invariant control) forward 5′-CCTCATATCCGGGGGAATGTG-3′ SEQ ID NO. 41) and reverse 5′-GCAGCAGCTGGCACCTTATTG-3′ (SEQ ID NO. 42).

### Immunofluorescence

For immunofluorescence, HeLa Kyoto cells were grown on coverslips (12 mm #1 VWR International) and synchronised as described above. The synchronised cells were rinsed in PBS, fixed in 1% PFA, permeabilised with 0.1% Triton X-100/0.02% SDS, and blocked in 2% BSA. After the blocking step, phosphohistone H3 (H3S10ph) antibody was applied for 1 h at a dilution of 1/500 and after three washes with PBS, Alexa Fluor 594 antibody was added at a dilution of 1/300 for 1 h. Coverslips were washed three times in PBS and mounted with Vectashield non-fade DAPI (Vector Laboratories).

### Cell Population Growth Assessment and Cell Cycle Analysis

Cell proliferation assessment and flow cytometric cell cycle analysis were performed as described in Rodriguez-Acebes supra.

### PAPPA and ZMPSTE24 Over-Expression in T47D Cells

PAPPA and ZMPSTE24 (control) are zinc-dependent metalloproteinases of the metzincin superfamily. Full-length human PAPPA cDNA (NM_{—}002581.3) and ZMPSTE24 cDNA (NM_{—}005857.2) were cloned into pCMV6-XL5 vectors (OriGene). Approximately 2×10⁶ T47D cells cultured in T75 flasks were transfected with 40 μg of PAPPA or ZMPSTE24 cDNA. Cells were collected 48 h and 72 h post-transfection. PAPPA and ZMPSTE24 expression levels were determined by qRT-PCR and western blot.

### RNAi-Rescue Experiments

Eight silent mutations were introduced into two separate sites of human PAPPA cDNA (NM_{—}002581.3) within the regions targeted by two different PAPP-A siRNAs (Ambion s10042 and 104028). The mutated cDNA was cloned into the pCMV6-XL5 vector (OriGene) and BT549 cells were transfected with 20 μg of the PAPPA rescue plasmid. Twenty-four hours after PAPPA+^{mut} overexpression, endogenous mRNA was knocked down using 100 nM PAPPA specific siRNA (Ambion s10042 or 104028). Cells were collected 48 h after knock down of endogenous mRNA.

### Invasion Assay

Invasion through extracellular matrix (ECMatrix) was measured in Boyden chamber assays (QCM Invasion Assay, Millipore) following the manufacturer's instructions. Briefly, BT549 cells were transfected with PAPPA ON-TARGETplus SMARTpool or control oligo in serum-free medium for 24 h. T47D cells were transfected with PAPPA or ZMPSTE24 expression constructs for 24 h prior starvation for 24 h in serum-free medium. BT549 and T47D cells were collected in RPMI medium containing 5% BSA, counted and 2.5×10⁵ cells were seeded in each invasion chamber. After incubation for 48 h (BT549) or 72 h (T47D) the invasion chamber inserts were washed with PBS, fixed in 4% paraformaldehyde for 5 min, stained with 0.1% crystal violet and cells from random areas on the filters were counted. Assays were performed in triplicate.

### β1-Integrin Cell Surface Expression

Live cells were immunostained in suspension (as described in Rizki, A. et al., J. Cancer research (2007) 67, 11106-11110), fixed in 2% PFA and FACS was performed as described in Rodriguez-Acebes et al, Am. J. Pathol, 2010; 177:2034-2045. The fluorescence peak was evaluated for its median value and corrected using samples, which had not been incubated with primary antibody (anti-CD29 HUTS-21, BD Pharmingen). To mask β1-integrin, 20 μg/ml of blocking antibody (anti-β1-integrin MAB1959, Chemicon; Vincourt, J. B. et al. (2010) Cancer research 70, 4739-4748) was added to the culture medium for the duration of the invasion assay.

### Statistical Analysis

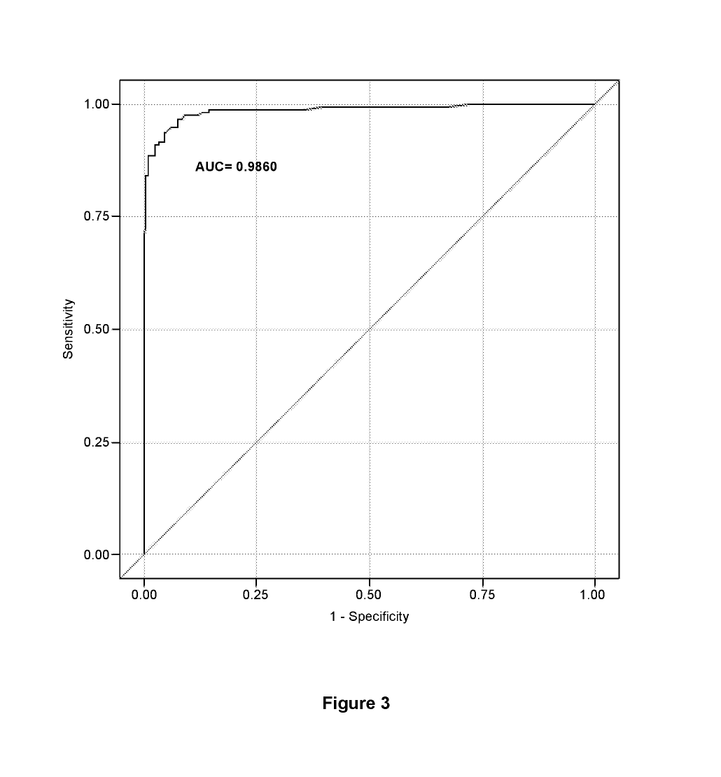

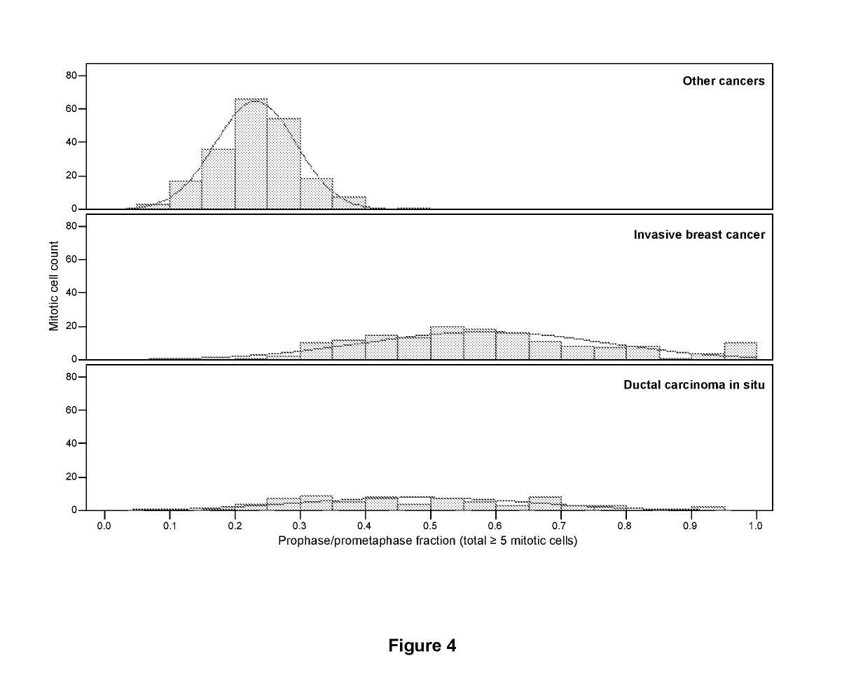

The proportion of mitotic cells in prophase/prometaphase was calculated for each specimen of premalignant and malignant tissue. Receiver Operating Characteristic (ROC) curves for differentiating breast cancer from other malignancies (pooled) using the proportion of cells in prophase/prometaphase were constructed for various minimum numbers of mitotic cells. It was clear that the ROC curve was not compromised by letting the minimum requirement be as low as five mitotic cells (FIG. 3). In the interest of using as many specimens as possible, the evaluability threshold for analysis of mitotic delay was set to at least five mitotic cells observed per specimen. A specimen was declared ‘delayed’ if at least one third of its mitotic cells were in prophase/prometaphase. This requirement was derived by balancing the sensitivity and specificity associated with distinguishing breast cancer from other malignancies: 94.9% of evaluable breast cancer specimens had at least one third of their mitotic cells in prophase/prometaphase, while 94.1% of evaluable other malignancies had less than one third of their mitotic cells in prophase/prometaphase (FIG. 4). The proportion of evaluable specimens with mitotic delay was compared between sources of specimen (breast cancer, DCIS, other malignancies) using Pearson's chi-squared test with Yates's continuity correction. The median proportion of mitotic cells in prophase/prometaphase was compared between sources of specimen using the Mann-Whitney test. All significance probabilities reported are two-sided.

The association of mitotic delay with tumour differentiation and nodal metastasis was assessed using a non-parametric Jonckheere-Terpstra test for trend, with morphological subtype and molecular subtype using a Krusal Wallis analysis of variance test, and with oestrogen/progesterone receptor status and aneuploidy using a Mann-Whitney test.

### Results

### Breast Cancer is Specifically Enriched in Early Mitotic Figures

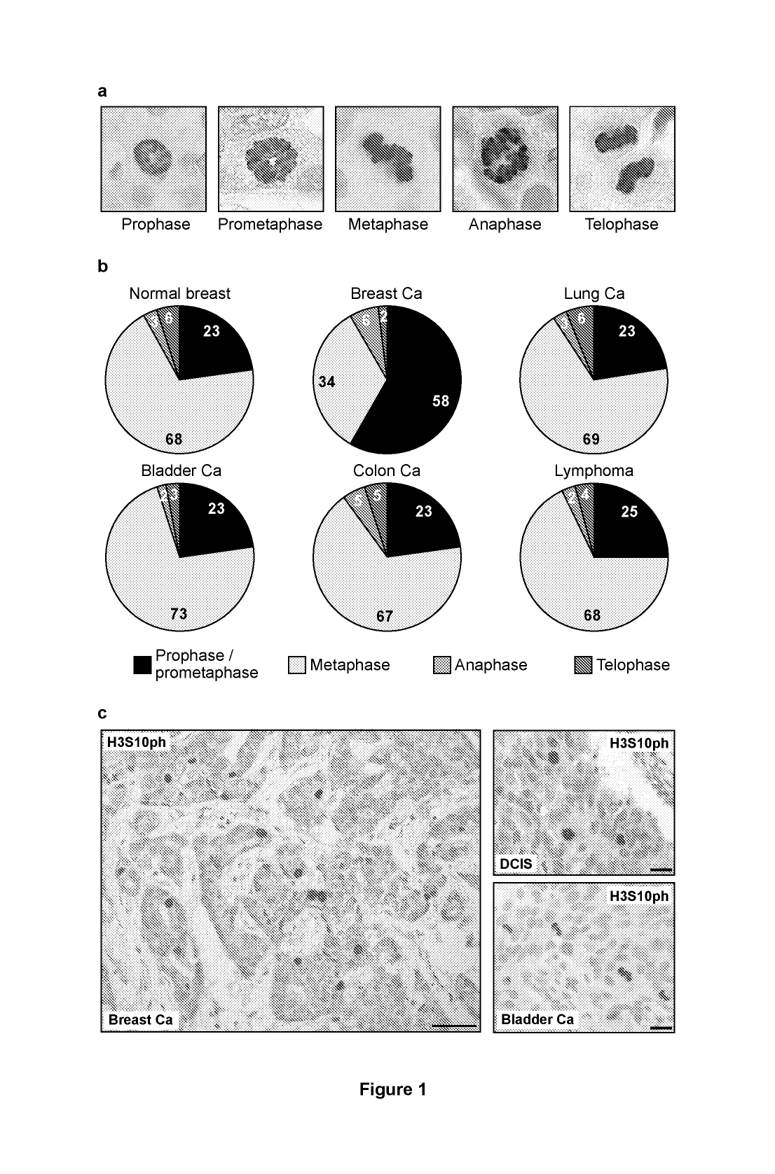

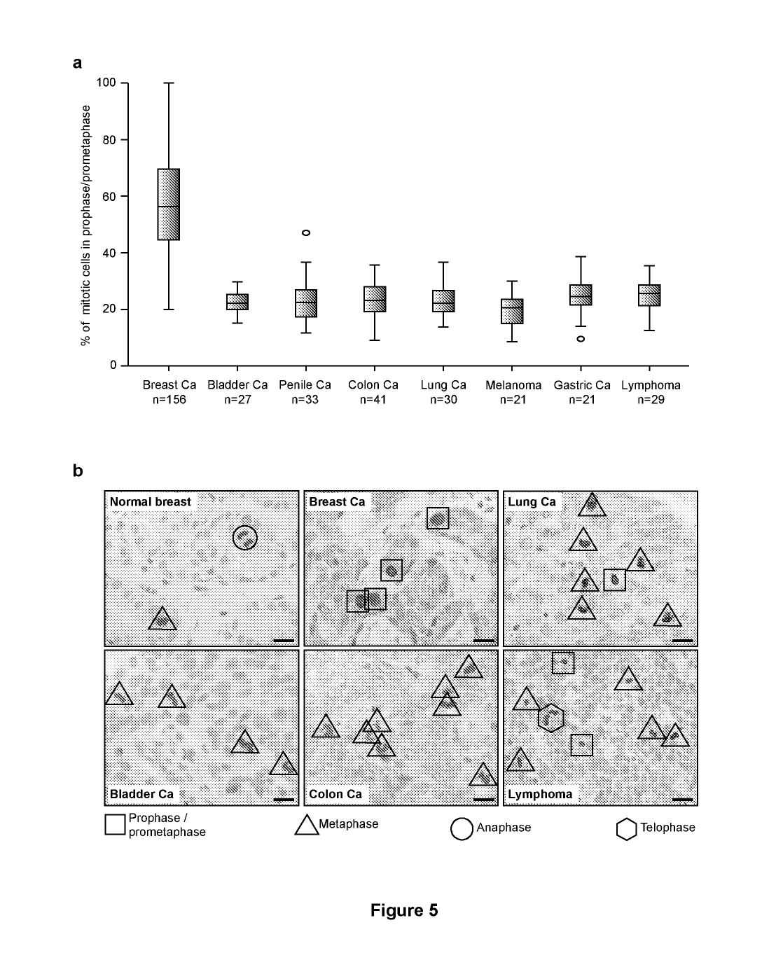

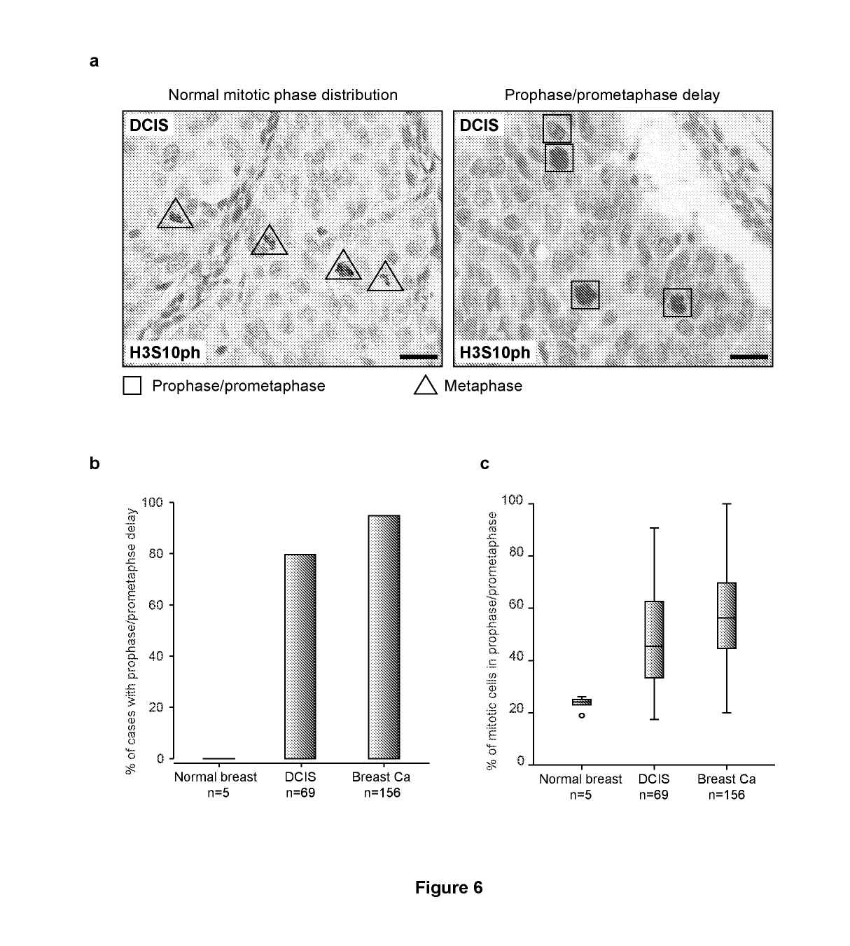

In evaluable tissue specimens from seven common human tumour types, including skin, lung, colon, gastric, bladder, penile and lymphatic cancer (n=202 patients), the majority of mitotic cells were in metaphase (FIG. 1a-b and FIG. 5a-b). In marked contrast the inventors found a strong prophase/prometaphase enrichment (defined as at least one third of mitotic cells in prophase/prometaphase) in 95% (148 out of 156 evaluable patients) of breast cancers compared with only 6% (12 out of 202) for the combined group of other malignancies (P<0.0001, Pearson's test with Yates's correction). The mean proportion of mitotic breast cancer cells in prophase/prometaphase was 58% (median 56%) compared with 23% (median 23%) in the other malignancies (P<0.0001, Mann-Whitney test) (FIG. 1b-c and FIG. 5a), indicating that an early mitotic delay or arrest is a hallmark of breast cancer. This was specific to diseased tissue, as normal proliferating breast tissue revealed an undisturbed mitotic phase distribution, again with 23% (median 24%) of mitotic cells residing in prophase/prometaphase (FIG. 1 b and FIG. 5b). Importantly, the inventors found could detect a clear mitotic delay phenotype already in 80% (55 out of 69 evaluable patients) of non-invasive ductal carcinoma in situ (DCIS) lesions (P<0.0001 compared with the group of other malignancies), in which the mean proportion of mitotic cells in prophase/prometaphase was 48% (median 45%) (P<0.0001 compared with the group of other malignancies) (FIG. 1 c and FIG. 6). Thus the tumour screen for specific mitotic phenotypes, first seen by gene silencing in a cell culture model (Neumann, B. et al. Nature (2010) 464, 721-727), identified an unexpectedly high frequency of early mitotic figures (prophase/prometaphase) in nearly all tested breast cancers, revealing a formerly unrecognized delay in mitotic progression in this tumour type.

MitoCheck Hits with a Breast Cancer-Like Mitotic Phenotype

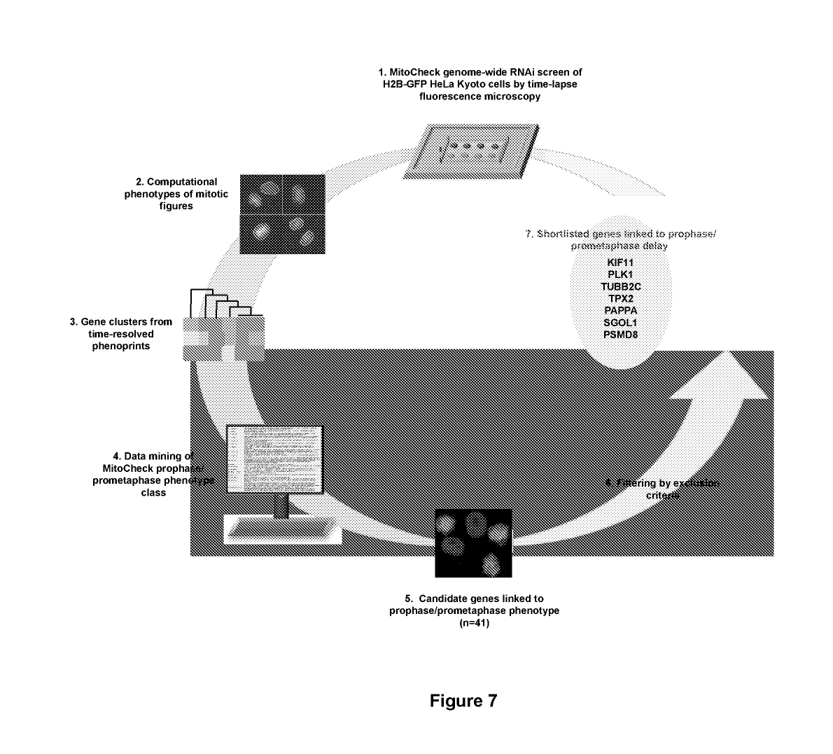

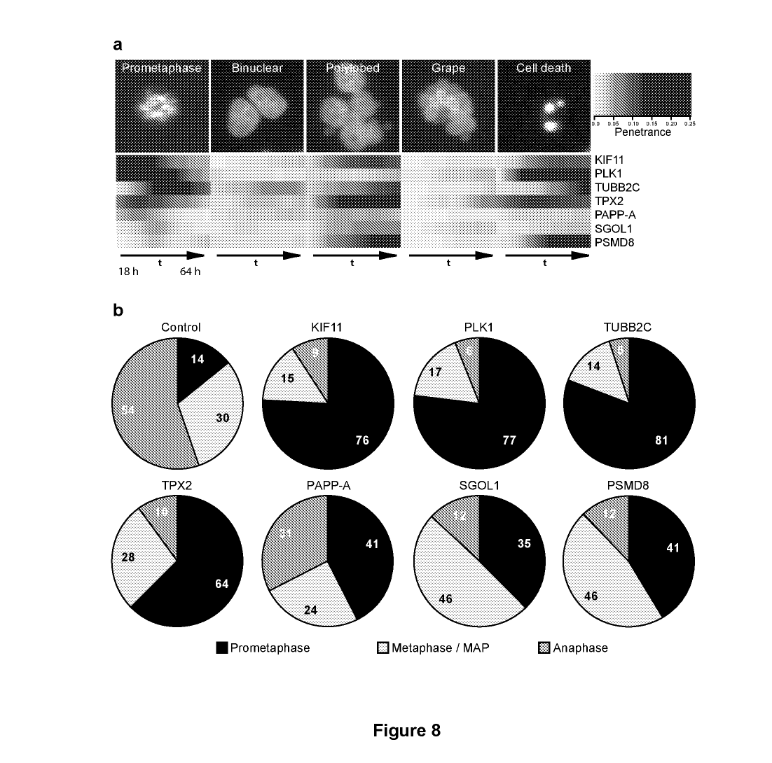

Early mitotic delay was a very specific and relatively rare mitotic phenotype in the MitoCheck screen (Neumann, B. et al.). In order to identify candidate genes whose down-regulation in cultured human cells results in a similar early mitotic phenotype to the one the inventors had observed in breast cancer, they searched the MitoCheck database (accessible at www.mitocheck.org), focusing specifically on the prophase/prometaphase class, which was morphologically most similar to the phenotypes that had been observed by the inventors in breast cancer tissues (FIG. 7). In the genome wide data set KIF11, PLK1, TUBB2C, TPX2, PAPPA, SGOL1 and PSMD8 displayed a significant increase in the prophase/prometaphase class (FIG. 8), indicating a delay or arrest in prophase/prometaphase, as detected in breast cancer. The quantitative scoring of the time-resolved phenotypic answers to the knock down of these individual genes showed for all seven genes as a primary phenotype prometaphase arrest, followed by secondary phenotypes (e.g. cell death or polylobed nuclear shape) (FIG. 8). RNAi experiments targeting KIF11, PLK1, TUBB2C and TPX2 showed a high percentage of cells in prometaphase already 12 to 25 hours after transfection, while PAPPA, SGOL1 and PSMD8 knock downs caused lower prometaphase phenotype penetrance but had an overall similar phenotypic profile (FIG. 8). Cell death as a consequence of the mitotic phenotype was significant for all genes, except for PAPPA and SGOL1 (FIG. 8), making them the strongest candidates for tumour suppressor genes whose mitotic aberrations are not expected to be cleared by cell death.

### PAPPA Loss is Linked to Mitotic Delay in Breast Cancer

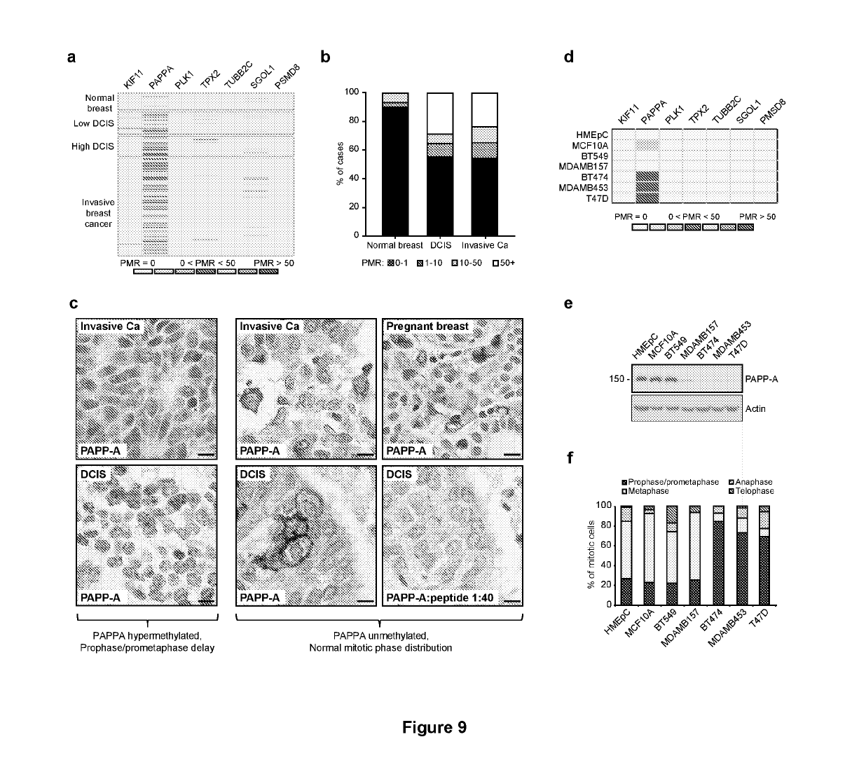

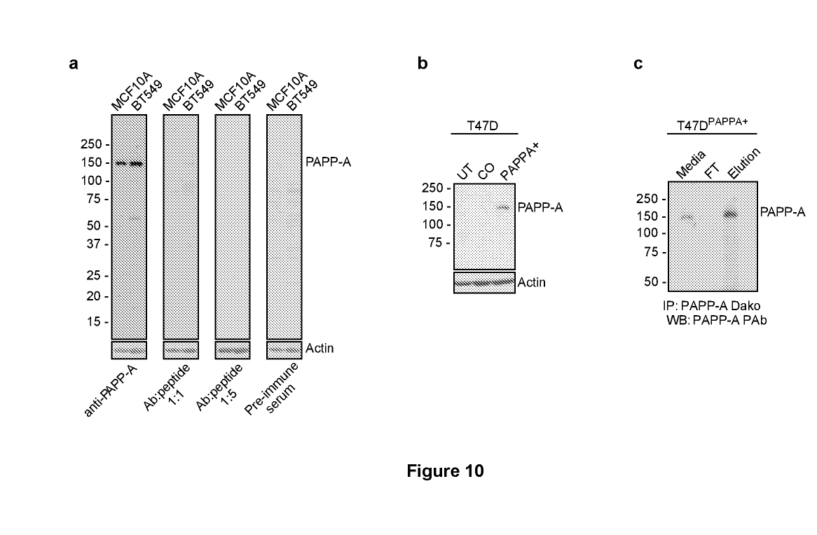

Since promoter methylation represents a common mechanism for loss-of-function of tumour suppressors during cancer development, the inventors hypothesised that epigenetic silencing of any of the seven MitoCheck candidate genes could be linked to the mitotic delay phenotype which the inventors found in breast cancer. Indeed, MethyLight assays (Widschwendter, M. et al. Cancer research (2004) 64, 3807-3813) showed that of the seven candidate genes only PAPPA is strongly hypermethylated in the 5′ regulatory region of the gene in invasive breast cancers and in non-invasive DCIS lesions (FIG. 9a). Forty-six % (80 out of 173 patients assessed for methylation) of breast cancers and 45% (34 out of 75 assessed patients) of DCIS lesions showed PAPPA hypermethylation (PMR>1; percentage methylated reference gene). In contrast, PAPPA was unmethylated in the majority of normal breast tissue samples (27 out of 30 assessed patients) (FIG. 9b; note that nine cases of breast cancer, six cases of DCIS and four cases of normal breast were not available for MethyLight analysis due to poor preservation of DNA). This made PAPPA the strongest candidate to explain the strong prophase/prometaphase delay found in breast cancer. To test if PAPPA promoter methylation indeed caused gene silencing, the inventors used a commercially available anti-PAPPA antibody (DAKO) for immuno-labelling of tissue sections and an affinity-purified rabbit anti-PAPPA PAb (FIG. 10) for western blotting. Immuno-expression analysis showed that indeed 96% (81 out of 84 patients) of non-invasive and invasive breast cancers with methylated PAPPA promoter and exhibiting the mitotic delay phenotype were not expressing PAPPA protein (FIG. 9c). By contrast, the majority of non-delayed/PAPPA unmethylated breast cancers (73%, 7 out of 11 patients), as well as normal proliferating pregnant breast tissue (n=5 patients) showed strong PAPPA immuno-staining predominantly at the cell membrane, consistent with a secreted protein (FIG. 9c).

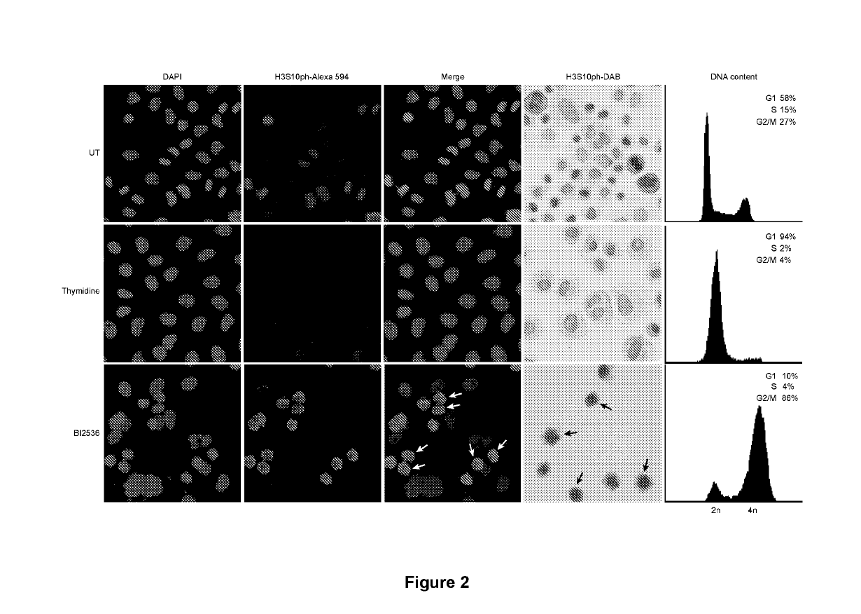

Validating the hypothesis that loss of PAPPA expression causes the mitotic delay in breast cancer requires an experimentally accessible system. The inventors therefore investigated whether the linkage between PAPPA gene silencing and the mitotic delay phenotype has been maintained in cultured breast cells. Consistent with the inventors' in vivo findings, cells with unmethylated PAPPA promoter and detectable PAPPA protein, including primary human mammary epithelial cells (HMEpC), immortalized MCF10A cells and the BT549 and MDAMB157 breast cancer cell lines, showed a normal mitotic phase distribution (FIG. 9d-f), as determined by morphological analysis of cytospin preparations immuno-labelled with the same phosphohistone H3 (H3S10ph) antibody used for the tissue screen (FIG. 2). By contrast, cell lines with heavily methylated PAPPA promoter (PMR>50) and strongly reduced PAPPA protein, including the BT474, MDAMB453 and T47D breast cancer cell lines, all exhibited the mitotic delay phenotype with ˜80% of mitotic cells residing in prophase/prometaphase (FIG. 9d-f).

### PAPPA is Required for Early Mitotic Progression in Breast Cancer Cells

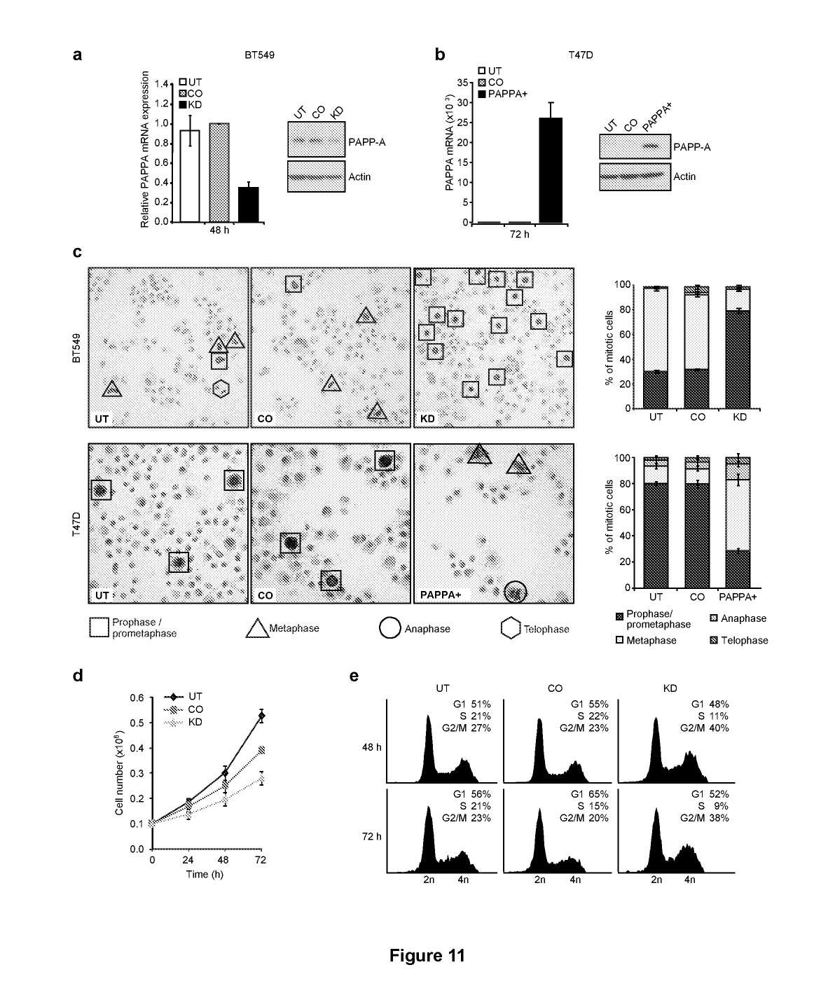

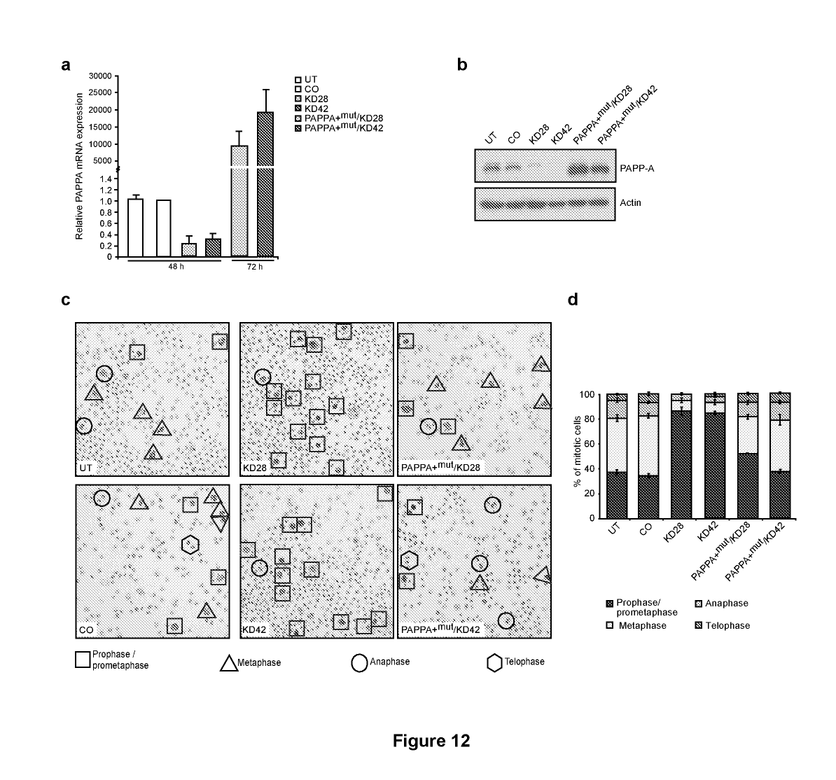

Having breast cancer cell lines in hand that recapitulate the link between loss of PAPPA expression and mitotic delay observed in patient tissue, the inventors first investigated whether PAPPA knock down by RNAi induces prophase/prometaphase delay in BT549 cells, in which the gene is not silenced through promoter methylation (FIG. 9e-f). Relative to control-siRNA, transfection of BT549 cells with a pool of four RNA duplexes targeting PAPPA mRNA reduced transcript levels by ˜70% and PAPP-A protein levels by ˜60% (FIG. 11a). Analysis of BT549 cytospin preparations immuno-labelled for phosphohistone H3 (H3S10ph) revealed that, indeed, PAPPA knock down induced a strong prophase/prometaphase delay phenotype in this cell line (FIG. 11c), very similar to the phenotype observed in HeLa cells in the MitoCheck screen (Neumann, B. et al. Nature (2010) 464, 721-727). Consistent with the increased proportion of mitotic BT549 cells in prophase/prometaphase, PAPPA knock down caused a marked increase in the cell population doubling time (FIG. 11d) with a concomitant ˜2-fold increase in the proportion of cells with G2/M DNA content (FIG. 11e) and a 3-fold increase in the mitotic index. The inventors verified the prophase/prometaphase delay phenotype caused by PAPP-A depletion by transfecting BT549 cells with single siRNA duplexes (siRNA-28 and siRNA-42) targeting different regions of the transcript (FIG. 12). To confirm that the RNAi phenotype was specifically due to PAPPA depletion, the inventors overexpressed a PAPPA cDNA variant resistant to siRNA-28 and siRNA-42. Expression of this construct restored PAPPA protein expression and rescued the mitotic delay phenotype caused by either of the two single siRNA duplexes (FIG. 12). If PAPPA loss causes the mitotic delay phenotype in breast cancer cells with hypermethylated PAPPA promoter such as T47D cells (FIG. 9d-e), the inventors reasoned that in this experimental system PAPPA overexpression should rescue the phenotype. Indeed, transfection of T47D cells with PAPPA cDNA (PAPPA+) restored PAPPA mRNA and protein levels (FIG. 11b) and fully reversed the mitotic delay phenotype, resulting in a mitotic phase distribution very similar to BT549 cells with normal PAPPA expression (FIG. 11c). Taken together, these results show that PAPPA is required for progression through early mitosis and that PAPPA down-regulation through epigenetic silencing (T47D cells) or experimentally by RNAi (BT549 cells) causes a strong prophase/prometaphase delay phenotype.

### PAPPA Loss Increases Invasiveness of Breast Cancer Cells

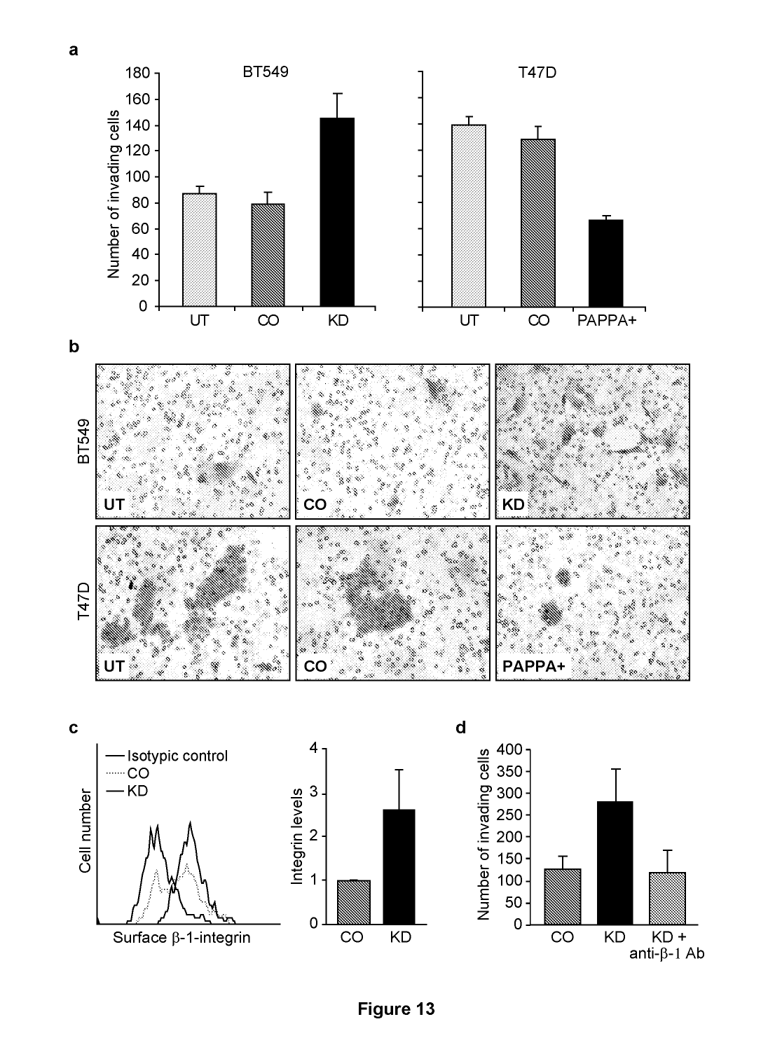

Next the inventors asked what biological advantage is conferred to the neoplastic breast cell through perturbation of early mitotic progression. To address this question they started by looking for any linkages between the mitotic delay phenotype and clinico-pathological features determined for each breast cancer specimen during routine clinical investigation (n=156 evaluable patients). This analysis revealed no linkage between mitotic delay and tumour differentiation (grade), morphological subtype (invasive ductal, lobular, mixed, mucinous, and micropapillary), molecular subtype (luminal, Her-2 or triple negative/basal-like), oestrogen/progesterone receptor status, nodal metastasis or aneuploidy. Notably, though, the presence of the mitotic delay phenotype in nearly all invasive breast cancer specimens studied (95%, 148 out of 156 evaluable patients) but in only a proportion of non-invasive DCIS lesions (80%, 55 out of 69) raises the possibility that this mitotic defect might be linked to the acquisition of invasiveness. To test this particular hypothesis, the inventors induced the mitotic delay phenotype in BT549 cells by PAPPA knock down (alternatively normal mitotic progression was restored in T47D cells by exogenous PAPPA expression) and measured the invasiveness of the manipulated cells in Matrigel-coated Boyden chamber assays. In parallel, the inventors used flow cytometry to determine the cell surface levels of β1-integrin, a well-characterised invasion marker in breast cancer. Silencing of PAPPA was associated with a marked (2-fold) increase in the number of invading cells (FIG. 13a-b). The increased invasiveness of PAPP-A depleted BT549 cells was also mirrored by an increase in β1-integrin cell surface levels (FIG. 13c). Notably, the increase in invasiveness associated with PAPPA knock down was fully reversed following treatment of BT549 cells with a β1-integrin blocking antibody prior to transfer of the cells to the Boyden chamber (FIG. 13d). Conversely, re-establishment of normal mitotic phase distribution by exogenous PAPPA expression strongly reduced the invasiveness of T47D cells (FIG. 13a-b). These results demonstrate that loss of PAPPA function delays progression through early mitosis and increases the capacity of breast cancer cells to become more invasive.

### Example 2

Exogenously added IGF-1 restores normal progression through mitosis in breast cancer cells displaying prophase/prometaphase delay phenotype.

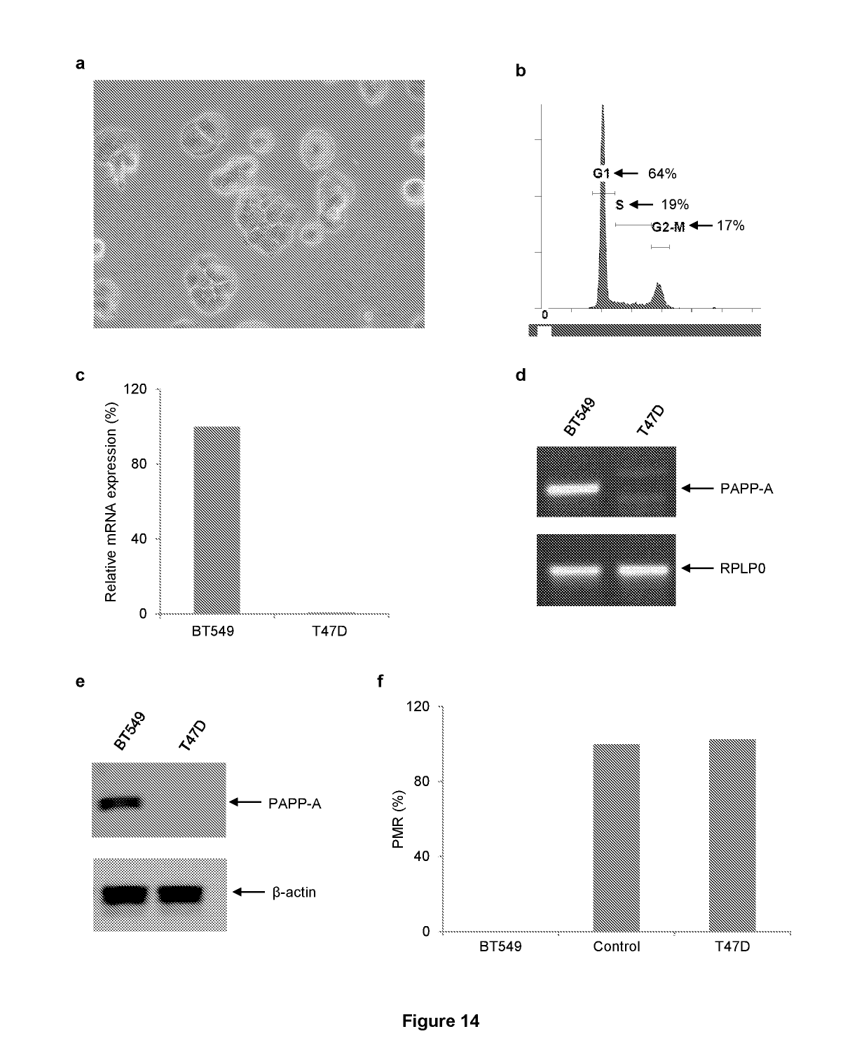

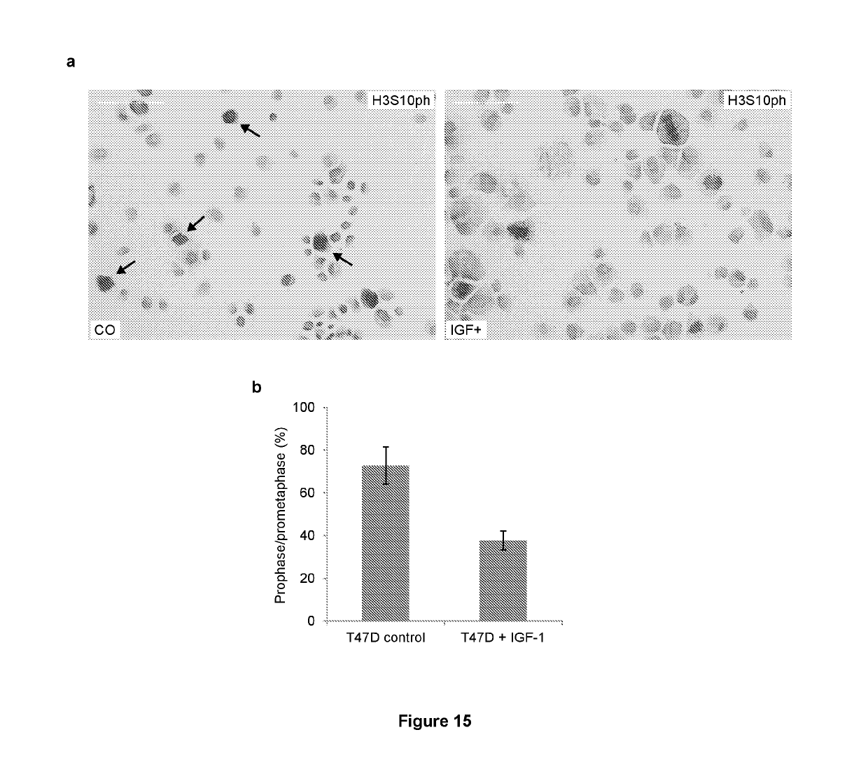

A known function of PAPPA is to release the hormone IGF-1 from its sequestering inhibitory binding protein IGFBP-4. By increasing the local bioavailability of IGF-1 at the cell surface, PAPPA activity increases IGF-dependent signalling and promotes cell growth and proliferation. Thus it can be postulated that PAPPA is likely to affect mitotic progression in breast cancer cells by modulating signalling through the IGF pathway. It follows from this that treatment of T47D breast cancer cells, which closely resemble tumour cells in vivo (i.e. display PAPPA promoter methylation and the mitotic delay phenotype; FIG. 14), with recombinant IGF-1 should restore normal progression through mitosis in this cell line. For these experiments, T47D cells were serum starved for 48 hours. The viability of the cells was established using Trypan Blue vital stain. The cells were treated with recombinant human IGF-1 (Imgenex) at a final concentration of 100 μM and harvested. Cells were harvested 24 hours later, re-suspended in PBS, cytospun onto glass slides and subjected to H3S10ph immuno-staining as described above. Indeed IGF-1 addition reduced the proportion of mitotic T47D cells in prophase/prometaphase by approximately one half (FIG. 15), close to levels seen in BT549 breast cancer cells expressing PAPPA. From this discovery a skilled person can derive that follow up treatment with an anti-mitotic agent will result in enhanced cancer cell killing. Thus sequential treatment with a first drug that removes the mitotic block and a second drug that targets mitosis after the prometaphase stage will chemosensitise tumour cells to the second drug.

### Methods

### Cell Culture

T47D cells were cultured in RPMI medium (ATCC) supplemented with 10% FCS and 10 μg/ml insulin. Cells were cultured at 37° C. with 5% CO₂ and ≧95% humidity. Cells were harvested following incubation with TrypLE™ Express (Life Technologies). Cell density and viability was determined by trypan blue exclusion using a Countess® Automated Cell Counter (Life Technologies). The population doubling time was calculated by PDT=(t2−t1)/3.32×(log n2−log n1), where t is the sampling time and n is the cell density at the time of sampling. Photomicrographs shown in FIG. 14A were taken using Panasonic digital camera fitted to the Leica inverted microscope.

### Cell Cycle Analysis

T47D cells that have reached 60-70% confluency were harvested by trypsinisation. The medium supernatant was pooled with the trypsinised cells and centrifuged for 3 minutes at 194×g. (This step was included to ensure retention of all mitotic cells and avoid loss through mitotic shake off). The cell pellet was washed and re-suspended in PBS to give a concentration of 2×10⁶ cells/ml. The re-suspended cells were then transferred to a Coulter Flow cytometry tube and fixed by the drop wise addition of 1.5 ml ice-cold 100% ethanol whilst vortexing. The cells were then placed on ice for 30 minutes to fix. Following ethanol fixation, the cells were pelleted by centrifugation for 5 minutes at 194×g. The supernatant was carefully removed and the cells washed with 2 ml PBS (added drop-wise whilst vortexing). The cells were finally re-suspended in 300 μl DNA Prep PI solution (Beckman Coulter) and incubated for 10 minutes at room temperature in the dark prior to cell cycle analysis on the Navios Flow Cytometer. Data was analysed using the Multicycle-AV software.

qRT-PCR Analysis

Total cellular RNA was isolated using the Ambion PureLink RNA Mini kit (Life Technologies), according to the manufacturer's instructions; qPCR reactions were carried out using the SuperscriptIII Platinum SYBR Green One-Step qRT-PCR kit (Life Technologies) according to the manufacturer's instructions. PCR reactions were carried out in StepOne Plus Real Time PCR system (Life Technologies). 400 ng template RNA and final primer concentration of 200 nM was used in each individual PCR reaction. PCR products were also subject to agarose gel electrophoresis as indicated in FIG. 14D.

Primers used in this study are indicated in Table 3 below:

TABLE 3

| | Forward | Reverse |
|---|---|---|
| | primer | primer |
| | sequence | sequence |
| Gene | 5'-3' | 5'-3' |
| PAPP-A | ACAGGCTACG | CTCACAGGCC |
| | TGCTCCAGAT | ACCTGCTTAT |
| | (SEQ ID | (SEQ ID |
| | NO. 39) | NO. 40) |
| RPLP0 | CCTCATATCC | GCAGCAGCTG |
| | GGGGGAATGTG | GCACCTTATTG |
| | (SEQ ID | (SEQ ID |
| | NO. 41) | NO. 42) |

### Immunoblotting

Protein concentration was determined using the Bradford protein assay kit (Pierce) according to the manufacturer's protocol. 20-30 μg of cytosolic protein and MagicMark™ (Life Technologies) were separated by Novex® 4-20% Tris-Glycine SDS PAGE (Life Technologies). Proteins were transferred from polyacrylamide gels onto PVDF membranes using the iBlot® dry electroblotting system (Life Technologies). Briefly, the membranes were blocked for 1 hour in PBS supplemented with 10% milk. The membrane was further probed with polyclonal anti-PAPP-A antibody (gift from academic collaborators). This step was carried out overnight at 4° C. with gentle agitation. After incubation with the primary antibody the membrane was washed five times for 10 minutes with PBS. HRP conjugated secondary goat anti-rabbit antibody (Dako) in PBS with 10% milk was added to the membrane and incubated for 1 hour at room temperature. Equal volumes of reagent A and B from ECLSelect™ kit (GE Healthcare) were added to the membrane and incubated for 3 min at room temperature. Image shown in FIG. 14E was captured using the GeneGnome chemiluminescent detection system. (Syngene). The membrane was re-probed with anti-β actin (Sigma Aldrich) antibody to ensure equal loading of total protein in each lane.

### Methylight Assay

Genomic DNA was extracted from T47D cells using the QIAamp DNA kit (Qiagen) according to the manufacturer's protocol. DNA isolated was quantified using NanoVue spectrophotometry. For each reaction 400-500 ng of genomic DNA was bisulfite-modified using the EZ DNA Methylation-Gold Kit (Zymo Research) according to the manufacturer's instructions. Bisulfite-modified DNA was stored at −80° C. until required. Unmodified SssI treated genomic DNA (New England Biolabs) was used as positive control. Two sets of primers and probes were designed for bisulfite-modified DNA: a methylated set for PAPP-A and collagen 2A1, to normalise for input DNA. qPCR reactions were carried out using the TaqMan® Universal PCR Master Mix, No AmpErase® UNG (Life technologies) with 20 ng DNA, 0.3 μM probe and 0.9 μM of both forward and reverse primer.

Primers used in this study are indicated in Table 4 below:

TABLE 4

| | Forward | Reverse | |
|---|---|---|---|
| | primer | primer | Probe |
| | sequence | sequence | sequence |
| Gene | 5'-3' | 5'-3' | 5'-3' |
| PAPP-A | GCGTGTT | CGCCTTC | 6-FAM- |
| | TGTGCGA | CGAATAT | TCGCCCG |
| | GAGTTGT | ACCCATT | AATATCT |
| | (SEQ ID | (SEQ ID | CTACGCCGCT- |
| | NO. 7) | NO. 8) | BHQ-1 |
| | | | (SEQ ID NO. 9) |
| COL2A1 | TCTAACA | GGGAAGA | 6-FAM- |
| | ATTATAA | TGGGATA | CCTTCATTCTAACC |
| | ACTCCAA | GAAGGGAATAT | CAATACCTATCCCA |
| | CCACCAA | (SEQ ID NO.32) | CCTCTAAA-BHQ-1 |
| | (SEQ ID | | (SEQ ID NO. 33) |
| | NO. 31) | | |

The reactions were carried out on a StepOne Plus Real Time PCR system (Life Technologies). The cycling conditions were: 95° C. (10 minutes), followed by 50 cycles of 95° C. (15 seconds), 60° C. (1 minute). The results of the qPCR reaction were analysed (DDC_{T} and RQ calculations) using the StepOne software. The percentage of fully methylated PAPP-A molecules was calculated by dividing the PAPP-A:COL2A1 ratio of the sample by the PAPP-A:COL2A1 ratio of the SssI-treated genomic DNA.

### IGF-1 Treatment of T47D Cells

T47D cells were serum starved for 48 hrs prior to the experiment. The viability of the cells was established using a vital stain Trypan blue using a Countess™ automated cell counter (Life technologies). The cells were treated with recombinant Human IGF-1 (IMR-233, Imgenex) at a final concentration of 100 μM. Cells were harvested 24 hrs later using Tryple Express™ (Life technologies) and re-suspended in PBS. The cells were then cytospun onto glass slides and subjected to H3S10ph immuno-staining as previously described.

### Results

The results are shown in FIGS. 14 and 15. Exogenous IGF-1 reverses the mitotic delay phenotype in T47D cells, thereby allowing the cells to undergo mitosis. Accordingly, chemotherapeutic agents that work during mitosis can be targeted to the cells to provide an effective therapy.

1. A method for determining the risk of progression of a proliferative lesion to invasive breast cancer and/or the risk of recurrent non-invasive disease in a patient, comprising detecting the presence and/or level of PAPPA in a breast tissue sample obtained from the patient, wherein if PAPPA is not present, or is present at a reduced level compared to a control, there is the risk of progression to invasive cancer and/or risk of recurrent disease.

2. A method for determining the risk of progression of a proliferative lesion to invasive breast cancer and/or the risk of recurrent non-invasive disease in a patient, comprising detecting the presence of loss of function-related genetic alterations in the PAPPA gene or its regulatory or promoter sequences in a sample obtained from the patient, wherein if genetic alterations are present there is the risk of progression to invasive cancer and/or risk of recurrent disease.

3. A method according to claim 2, wherein loss of function-related genetic alteration in the PAPPA gene or its regulatory or promoter sequences is methylation.

4. A method according to claim 1, wherein the presence of PAPPA is identified using a PAPPA-specific antibody or a probe for the PAPPA gene, mRNA or specific PAPPA mutations.

5. A method for determining the risk of progression of a proliferative lesion to invasive breast cancer and/or the risk of recurrent non-invasive disease in a patient, comprising identifying the proportion of mitotic cells in a breast tissue sample obtained from the patient that are in prophase or pro-metaphase and comparing to a pre-determined cut-off value, wherein if the proportion of cells in prophase or pro-metaphase is greater than the cut-off value there is risk of progression to invasive breast cancer and/or risk of recurrent disease.

6. A method according to claim 5, wherein the cut-off value is at least 30% of the mitotic cells in the sample.

7. A method according to claim 5, wherein at least five of the cells in the tissue sample are in mitosis.

8. A method according to claim 5, wherein the proportion of cells that are in prophase or pro-metaphase is determined using immuno-detection.

9. A method according to claim 8, wherein immuno-detection is carried out using an H3S10ph antibody.

10. A method according to claim 1, wherein the tissue sample is breast tissue that exhibits proliferative lesions.

11. A method according to claim 10, wherein the proliferative lesions are selected from pre-invasive lesions including ductal carcinoma-in-situ (DCIS), lobular carcinoma-in-situ (LCIS) and Paget's disease of the nipple and proliferative lesions with uncertain malignant potential, including lobular neoplasia, lobular intraepithelial neoplasia, atypical lobular hyperplasia (ALH), flat epithelial atypia (FEA), atypical ductal hyperplasia (ADH) microinvasive carcinoma, intraductal papillary neoplasms and phyllodes tumour.

12. A method according to claim 5, further comprising detecting the presence and/or level of PAPPA in a breast tissue sample obtained from the patient, wherein if PAPPA is not present, or is present at a reduced level compared to a control, there is the risk of progression to invasive breast cancer and/or risk of recurrent disease.

13. The method according to claim 12, wherein the presence of PAPPA is identified using a PAPPA-specific antibody.

14. The method according to claim 5, when pendant on claim 5, further comprising detecting the presence of methylation of the PAPPA gene, or its regulatory or promoter sequences, in a breast tissue sample obtained from the patient, wherein if methylation is present, there is the risk of progression to invasive breast cancer and/or risk of recurrent disease.

15. (canceled)

16. A therapeutic regimen for treating or preventing breast cancer in a patient, comprising:
i. administering a first drug which releases mitotically delayed cells; and
ii. sequentially administering a second drug which is a chemotherapeutic agent.

17. A therapeutic regimen according to claim 16, wherein the first drug is PAPPA protein, or a nucleic acid encoding functional PAPPA, an IGF receptor agonist (e.g. IGF-1) or a demethylation agent.

18. A therapeutic regimen according to claim 16, wherein the second drug is selected from the group comprising taxanes and vinca alkaloids.

19. A therapeutic regimen according to claim 16, wherein the patient is initially identified as having the mitotic delay phenotype by:
i. identifying the proportion of mitotic cells in a breast tissue sample obtained from the patient that are in prophase or pro-metaphase; and
ii. comparing to a pre-determined cut-off value,
wherein the mitotic delay phenotype is identified if the proportion of cells in prophase or pro-metaphase is greater than the cut-off value, and wherein the tissue sample contains at least five mitotic cells.

20. A therapeutic regimen according to claim 19, wherein the cut-off value is at least 30% of mitotic cells within the breast tissue sample.

21. A therapeutic regimen according to claim 1, wherein the patient is initially identified as being a suitable candidate for treatment according to the therapeutic regimen by detecting PAPPA loss or the presence of loss of function-related genetic alterations in the PAPPA gene or its regulatory or promoter sequences in a sample obtained from the patient, wherein if PAPPA loss or genetic alterations are present the patient is identified as being a suitable candidate for treatment according to the therapeutic regimen.

22. A therapeutic regimen according to claim 21, wherein PAPPA loss or the loss of function-related genetic alteration in the PAPPA gene or its regulatory or promoter sequences is due to methylation.

23. A chemotherapeutic agent that targets molecular events during cell division, for use in the treatment or prevention of breast cancer, wherein the chemotherapeutic is to be administered to a patient who has been prior treated with a molecule that releases a cell from mitotic delay.

24. A chemotherapeutic agent for use according to claim 23, wherein the chemotherapeutic agent is selected from the group comprising taxanes and vinca alkaloids.

25. A chemotherapeutic agent for use according to claim 23, wherein the molecule is a demethylation agent, an IGF receptor agonist, preferably IGF-1, or PAPPA protein.


SEQUENCE LISTING (42 sequences)

SEQ ID NO: 1 - DNA, 21 bp, Artificial
  Synthetic primer sequence
cgagcgttgtatgttgggatt

SEQ ID NO: 2 - DNA, 22 bp, artificial
  Synthetic primer sequence
cgcaacgaacgataacatctca

SEQ ID NO: 3 - DNA, 20 bp, Artificial
  Synthetic probe sequence
aactacgcaaacatccgccg

SEQ ID NO: 4 - DNA, 24 bp, Artificial
  Synthetic primer sequence
gcgtcgaggtttttaaagttggta

SEQ ID NO: 5 - DNA, 21 bp, Artificial
  Synthetic primer sequence
cccaactccaaaaccgcatat

SEQ ID NO: 6 - DNA, 19 bp, Artificial
  Synthetic probe sequence
ccctacaccgccacccgaa

SEQ ID NO: 7 - DNA, 21 bp, Artificial
  Synthetic primer sequence
gcgtgtttgtgcgagagttgt

SEQ ID NO: 8 - DNA, 21 bp, Artificial
  Synthetic primer sequence
cgccttccgaatatacccatt

SEQ ID NO: 9 - DNA, 24 bp, Artificial
  Synthetic probe sequence
tcgcccgaatatctctacgccgct

SEQ ID NO: 10 - DNA, 20 bp, Artificial
  Synthetic primer sequence
gttcgggcgttcgtgttaat

SEQ ID NO: 11 - DNA, 17 bp, Artificial
  Synthetic primer sequence
gccgcgcaacaccataa

SEQ ID NO: 12 - DNA, 28 bp, Artificial
  Synthetic probe sequence
ccctacgcaacaacaacctttaaacccg

SEQ ID NO: 13 - DNA, 23 bp, Artificial
  Synthetic primer sequence
ggttttatcggcgaaagagattt

SEQ ID NO: 14 - DNA, 17 bp, Artificial
  Synthetic primer sequence
cgaccccgcacataacg

SEQ ID NO: 15 - DNA, 26 bp, Artificial
  Synthetic probe sequence
ccgactacgtaaatccactaaaacct

SEQ ID NO: 16 - DNA, 23 bp, Artificial
  Synthetic primer sequence
cgcgtttcgttgttagatttgag

SEQ ID NO: 17 - DNA, 20 bp, Artificial
  Synthetic primer sequence
aaaaacccgcgccctaacta

SEQ ID NO: 18 - DNA, 24 bp, Artificial
  Synthetic probe sequence
cttcccacgactcacctaacctcg

SEQ ID NO: 19 - DNA, 22 bp, Artificial
  Synthetic primer sequence
tggcgataggattttgttgtga

SEQ ID NO: 20 - DNA, 21 bp, Artificial
  Synthetic primer sequence
gacaacctcccgcaactcttt

SEQ ID NO: 21 - DNA, 22 bp, Artificial
  Synthetic probe sequence
tttcctccgtttcccgaacgaa

SEQ ID NO: 22 - DNA, 22 bp, Artificial
  Synthetic primer sequence
aacgaacgcgcaaaatactaca

SEQ ID NO: 23 - DNA, 28 bp, Artificial
  Synthetic primer sequence
cgttttggtagtttatttcgagattagc

SEQ ID NO: 24 - DNA, 21 bp, Artificial
  Synthetic probe sequence
ccgacgctccgccaacgatta

SEQ ID NO: 25 - DNA, 19 bp, Artificial
  Synthetic primer sequence
cgcacattcgctcaaatcc

SEQ ID NO: 26 - DNA, 22 bp, Artificial
  Synthetic primer sequence
cgtcgagattcgatcgtaggtt

SEQ ID NO: 27 - DNA, 29 bp, Artificial
  Synthetic probe sequence
catccgaacatccacctaaccctaaaacg

SEQ ID NO: 28 - DNA, 21 bp, Artificial
  Synthetic primer sequence
ttcgtcgggtaagcgtttgta

SEQ ID NO: 29 - DNA, 20 bp, Artificial
  Synthetic primer sequence
gcgaccgccatcttacgtaa

SEQ ID NO: 30 - DNA, 26 bp, Artificial
  Synthetic probe sequence
cggcgttgtcgtaaattaggcggttt

SEQ ID NO: 31 - DNA, 28 bp, Artificial
  Synthetic primer sequence
tctaacaattataaactccaaccaccaa

SEQ ID NO: 32 - DNA, 25 bp, Artificial
  Synthetic primer sequence
gggaagatgggatagaagggaatat

SEQ ID NO: 33 - DNA, 36 bp, Artificial
  Synthetic probe sequence
ccttcattctaacccaatacctatcccacctctaaa

SEQ ID NO: 34 - DNA, 23 bp, Artificial
  Synthetic primer sequence
tccgtaagtgcagcttctctttg

SEQ ID NO: 35 - DNA, 20 bp, Artificial
  Synthetic primer sequence
tggagcccacaactgtcaga

SEQ ID NO: 36 - DNA, 34 bp, Artificial
  Synthetic probe sequence
caaagtacagagtcaagagttccaaagccacaga

SEQ ID NO: 37 - RNA, 19 nt, Artificial
  Synthetic primer sequence
gagccuacuuggauguuaa

SEQ ID NO: 38 - RNA, 19 nt, Artificial
  Synthetic primer sequence
uuaacauccaaguaggcuc

SEQ ID NO: 39 - DNA, 20 bp, Artificial
  Synthetic primer sequence
acaggctacgtgctccagat

SEQ ID NO: 40 - DNA, 20 bp, Artificial
  Synthetic primer sequence
ctcacaggccacctgcttat

SEQ ID NO: 41 - DNA, 21 bp, Artificial
  Synthetic primer sequence
cctcatatccgggggaatgtg

SEQ ID NO: 42 - DNA, 21 bp, Artificial
  Synthetic primer sequence
gcagcagctggcaccttattg